(19) United States
(12) Patent Application Publication
(10) Pub. No.: US 2023/0121320 A1
(43) Pub. Date: Apr. 20, 2023

(54) STING AGONIST COMBINATION TREATMENTS WITH CYTOKINES

(71) Applicants: IMMUNESENSOR THERAPEUTICS, INC., Dallas, TX (US); THE BOARD OF REGENTS OF THE UNIVERSITY OF TEXAS SYSTEM, Austin, TX (US)

(72) Inventors: Zhijian CHEN, Dallas, TX (US); Lijun SUN, Dallas, TX (US); Youtong WU, Dallas, TX (US); Huiling TAN, Dallas, TX (US)

(21) Appl. No.: 17/814,824

(22) Filed: Jul. 25, 2022

Related U.S. Application Data
(60) Provisional application No. 63/203,481, filed on Jul. 23, 2021.

(51) Int. Cl. A61K 38/20; A61K 31/7052; A61P 35/00

(52) U.S. Cl. CPC A61K 38/208; A61K 31/7052; A61P 35/00

(57) ABSTRACT

The disclosure provides, among other things, methods and uses for treating a disease or disorder, particularly tumors of a cancer patient, comprising conjointly administering effective amounts of a STING agonist, a cytokine, and an optional immune checkpoint inhibitor to the patient, wherein the STING agonist or the cytokine is intratumorally administered to the patient.

### 1. SEQUENCE LISTING

The instant application contains a Sequence Listing with three sequences which has been submitted via USPTO Patent Center and are hereby incorporated by reference in its entirety. Said XML copy, created on Jul. 18, 2022, is named “39143-52882 008US_Sequence Listing.xml” and is 14 kilobytes in size.

### 2. FIELD

This disclosure pertains to, among other things, the use of agonists of STimulator of INterferon Genes (STING) in combination with cytokines for activating the immune system to treat certain diseases or disorders, including cancer. This disclosure also pertains to the use of a STING agonist (such as a cyclic dinucleotide), a cytokine (such as an interleukin), and an immune checkpoint inhibitor to treat certain diseases or disorders, including cancer.

### 3. BACKGROUND

The treatment of advanced solid tumor malignancies as well as many hematologic malignancies continues to be defined by high unmet medical need. In most settings, treatment with cytotoxic chemotherapy and targeted kinase inhibitors leads to the emergence of drug-resistant tumor clones and subsequent tumor progression and metastasis.

In recent years, notable success has been achieved through alternate approaches oriented around activation of immune-mediated tumor destruction. The immune system plays a pivotal role in defending humans and animals against cancer. The anti-tumor effect is controlled by positive factors that activate anti-tumor immunity and negative factors that inhibit the immune system. Negative factors that inhibit anti-tumor immunity include immune checkpoint proteins, such as cytotoxic T-lymphocyte-associated protein 4 (CTLA-4), programmed cell death 1 (PD-1), and programmed death-ligand 1 (PD-L1). Immuno-oncology (IO) approaches, including antibodies against these checkpoint proteins, have shown remarkable efficacy in several types of human cancers.

However, existing cancer immunotherapy through immune checkpoint blockade is effective for only a small fraction (on average 20-30%) of cancer patients. The patients who are refractory to immune checkpoint blockade often have tumors that are not inflamed, or so-called “cold” tumor cells, i.e., they lack tumor-infiltrating leukocytes (TILs), such as cluster of differentiation 8 (CD8) T cells, or the tumor microenvironment suppresses the functions of the TILs. A major thrust of ongoing cancer drug development research remains focused on transforming “cold” tumor cells into “hot” tumor cells in order to achieve better tumor control across a wider array of patients.

The innate immune system, which is the first line of defense against pathogens and cancer cells, is important for turning the non-inflamed tumors (“cold”) into an inflamed (“hot”) microenvironment. A recently discovered innate immunity pathway, the cGAS-STING pathway (involving the protein Cyclic GMP-AMP Synthase (cGAS)), plays a critical role in anti-tumor immunity. cGAS is a DNA sensing enzyme that activates the type-I interferon pathway. Upon binding to DNA, cGAS is activated to synthesize the cyclic dinucleotide (CDN) 2′3′-cyclic-GMP-AMP (2′3′-cGAMP), which then functions as a secondary messenger that binds to and activates the adaptor protein STING. STING then activates a signal transduction cascade leading to the production of type-I interferons, cytokines, and other immune mediators.

While cytokine production is essential for generating anti-tumor immunity, high cytokines levels pose a safety concern. Specifically, high cytokine levels can evoke a dangerous inflammatory response in cancer patients undergoing immunotherapy, thereby discouraging the use of cytokines in IO applications. Selection of the type and amount of an appropriate cytokine to leverage its anti-tumor effect while reducing or limiting its systemic toxicity has remained a challenging unmet need. As a result, there remains a significant unmet medical need to develop therapies that can provoke specific and systemic immune responses to tumors throughout the body, including those tumors that are not or cannot be treated directly (i.e., through an abscopal effect), such as due to their location or size.

### 4. SUMMARY

The disclosure provides methods of administering STING agonists to patients, such as human cancer patients, in combination with cytokines, and optionally in further combination with one or more immune checkpoint inhibitors, such as inhibitors of CTLA-4, PD-I, and/or PD-L1, particularly antibody inhibitors of these proteins. The present disclosure also provides combination therapies capable of use in such methods and treatments.

In one aspect, the disclosure provides a method of treating tumors in a cancer patient in need thereof, comprising conjointly administering effective amounts of a STING agonist and a cytokine to the patient, wherein the STING agonist or the cytokine is intratumorally administered to the patient. In certain embodiments, both the STING agonist and the cytokine are administered intratumorally to the patient. In one particular aspect, the disclosure provides a method of treating tumors in a cancer patient in need thereof, comprising conjointly administering effective amounts of a STING agonist and a cytokine to the patient, wherein the STING agonist or the cytokine is intratumorally administered to the patient and wherein the patient exhibits reduced recurrence of the tumors following treatment, including in the absence of further treatment. In certain of these embodiments, both the STING agonist and the cytokine are administered intratumorally to the patient

In particular embodiments, the disclosure provides a method of treating tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically administering an effective amount of an immune checkpoint inhibitor (such as an anti-PD-1 antibody, an anti-PD-L1 antibody, or an anti-CTLA-4 antibody) to the cancer patient.

In other particular embodiments, the disclosure provides a method of treating tumors in a cancer patient in need thereof, comprising conjointly administering effective amounts of a STING agonist (such as a CDN), a cytokine (such as an interleukin), and an immune checkpoint inhibitor (such as an anti-PD-I antibody, an anti-PD-L1 antibody, or an anti-CTLA-4 antibody) to the patient, wherein both the STING agonist and the cytokine are administered intratumorally to the patient, and the immune checkpoint inhibitor is administered systemically to the cancer patient.

In another aspect, the disclosure provides a method of augmenting the anti-tumor response of a cancer patient, comprising conjointly administering effective amounts of a STING agonist and a cytokine to the patient, wherein the STING agonist or the cytokine is intratumorally administered to the patient. In certain embodiments, both the STING agonist and the cytokine are administered intratumorally to the patient.

In particular embodiments, the disclosure provides a method of augmenting the anti-tumor response of a cancer patient, comprising conjointly administering effective amounts of a STING agonist and a cytokine to the patient, wherein both the STING agonist and the cytokine are administered intratumorally to the patient, and the method further comprises conjointly systemically administering an effective amount of an immune checkpoint inhibitor (such as an anti-PD-1 antibody, an anti-PD-L1 antibody, or an anti-CTLA-4 antibody) to the cancer patient.

In other particular embodiments, the disclosure provides a method of augmenting the anti-tumor response of a cancer patient, comprising conjointly administering effective amounts of a STING agonist (such as a CDN), a cytokine (such as an interleukin), and an immune checkpoint inhibitor (such as an anti-PD-I antibody, an anti-PD-L1 antibody, or an anti-CTLA-4 antibody) to the patient, wherein both the STING agonist and the cytokine are administered intratumorally to the patient, and the immune checkpoint inhibitor is administered systemically to the cancer patient.

In yet another aspect, the disclosure provides a method of increasing the population or function of immune cells (such as T cells, NK cells, B cells, dendritic cells, or macrophages, or a combination thereof) of a cancer patient, comprising conjointly administering effective amounts of a STING agonist and a cytokine to the patient, wherein the STING agonist or the cytokine is intratumorally administered to the patient. In certain embodiments, both the STING agonist and the cytokine are administered intratumorally to the patient.

In particular embodiments, the disclosure provides a method of increasing the population or function of immune cells (such as T cells, NK cells, B cells, dendritic cells, or macrophages, or a combination thereof) of a cancer patient, comprising conjointly administering effective amounts of a STING agonist and a cytokine to the patient, wherein both the STING agonist and the cytokine are administered intratumorally to the patient, and the method further comprises conjointly systemically administering an effective amount of an immune checkpoint inhibitor (such as an anti-PD-1 antibody, an anti-PD-L1 antibody, or an anti-CTLA-4 antibody) to the cancer patient.

In other particular embodiments, the disclosure provides a method of increasing the population or function of immune cells (such as T cells, NK cells, B cells, dendritic cells, or macrophages, or a combination thereof) of a cancer patient, comprising conjointly administering effective amounts of a STING agonist (such as a CDN), a cytokine (such as an interleukin), and an immune checkpoint inhibitor (such as an anti-PD-1 antibody, an anti-PD-L1 antibody, or an anti-CTLA-4 antibody) to the patient, wherein both the STING agonist and the cytokine are administered intratumorally to the patient, and the immune checkpoint inhibitor is administered systemically to the cancer patient.

In yet another aspect, the disclosure provides a method of reducing recurrence of tumors in a cancer patient in need thereof, comprising, conjointly administering effective amounts of a STING agonist and a cytokine to the patient, wherein the STING agonist or the cytokine is intratumorally administered to the patient. In certain embodiments, both the STING agonist and the cytokine are administered intratumorally to the patient.

In particular embodiments, the disclosure provides a method of reducing recurrence of tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically administering an effective amount of an immune checkpoint inhibitor (such as an anti-PD-1 antibody, an anti-PD-L1 antibody, or an anti-CTLA-4 antibody) to the cancer patient.

In other particular embodiments, the disclosure provides a method of reducing recurrence of tumors in a cancer patient in need thereof, comprising conjointly administering effective amounts of a STING agonist (such as a CDN), a cytokine (such as an interleukin), and an immune checkpoint inhibitor (such as an anti-PD-1 antibody, an anti-PD-L1 antibody, or an anti-CTLA-4 antibody) to the patient, wherein both the STING agonist and the cytokine are administered intratumorally to the patient, and the immune checkpoint inhibitor is administered systemically to the cancer patient.

In another aspect, the disclosure provides a method of treating of tumors in a cancer patient in need thereof, comprising causing a STING agonist (such as a CDN), a cytokine (such as an interleukin), and an immune checkpoint inhibitor (such as an anti-PD-1 antibody, an anti-PD-L1 antibody, or an anti-CTLA-4 antibody) to be concurrently present in the patient's body.

In a particular embodiment, the method comprises administering an effective amount of the STING agonist to the patient, wherein the patient has already been administered the cytokine and the immune checkpoint inhibitor. In another particular embodiment, the method comprises administering an effective amount of the cytokine to the patient, wherein the patient has already been administered the STING agonist and the immune checkpoint inhibitor. In yet another particular embodiment, the method comprises administering an effective amount of the immune checkpoint inhibitor to the patient, wherein the patient has already been administered the STING agonist and the cytokine.

In another aspect, the disclosure provides a method of reducing recurrence of tumors in a patient, comprising causing a STING agonist (such as a CDN), a cytokine (such as an interleukin), and an immune checkpoint inhibitor (such as an anti-PD-1 antibody, an anti-PD-L1 antibody, or an anti-CTLA-4 antibody) to be concurrently present in the patient's body. In a particular embodiment, the method comprises administering an effective amount of the STING agonist to the patient, wherein the patient has already been administered the cytokine and the immune checkpoint inhibitor. In another particular embodiment, the method comprises administering an effective amount of the cytokine to the patient, wherein the patient has already been administered the STING agonist and the immune checkpoint inhibitor. In yet another particular embodiment, the method comprises administering an effective amount of the immune checkpoint inhibitor to the patient, wherein the patient has already been administered the STING agonist and the cytokine.

In another aspect, the disclosure provides a method of preventing recurrence of tumors in a patient, comprising causing a STING agonist (such as a CDN), a cytokine (such as an interleukin), and an immune checkpoint inhibitor (such as an anti-PD-1 antibody, an anti-PD-L1 antibody, or an anti-CTLA-4 antibody) to be concurrently present in the patient's body. In a particular embodiment, the method comprises administering an effective amount of the STING agonist to the patient, wherein the patient has already been administered the cytokine and the immune checkpoint inhibitor. In another particular embodiment, the method comprises administering an effective amount of the cytokine to the patient, wherein the patient has already been administered the STING agonist and the immune checkpoint inhibitor. In yet another particular embodiment, the method comprises administering an effective amount of the immune checkpoint inhibitor to the patient, wherein the patient has already been administered the STING agonist and the cytokine.

In a further aspect, the disclosure provides a combination therapy, such as for treating tumors in a cancer patient in need thereof, comprising a STING agonist and a cytokine, wherein the STING agonist or the cytokine is formulated for intratumoral administration to the patient. In certain embodiments, both the STING agonist and the cytokine are formulated for intratumoral administration to the patient.

In particular embodiments, the disclosure provides a combination therapy, such as for treating tumors in a cancer patient in need thereof, wherein both the STING agonist and the cytokine are formulated for intratumoral administration to the patient, and the combination therapy further comprises an immune checkpoint inhibitor (such as an anti-PD-I antibody, an anti-PD-L1 antibody, or an anti-CTLA-4 antibody) formulated for systemic administration to the cancer patient.

In some embodiments, the disclosure provides a mixture comprising a STING agonist, a cytokine, and an immune checkpoint inhibitor. In some embodiments the mixture further comprises human plasma.

In particular embodiments, the disclosure provides a mixture comprising a STING agonist that is a CDN; a cytokine that is an interleukin; an immune checkpoint inhibitor that is an anti-PD-1 antibody, an anti-PD-L1 antibody, or an anti-CTLA-4 antibody; and human plasma.

In particular embodiments, the disclosure provides a method of treating tumors in a cancer patient in need thereof, comprising conjointly administering effective amounts of a STING agonist and a cytokine to the patient, wherein the STING agonist or the cytokine is intratumorally administered to the patient and wherein the patient exhibits reduced recurrence of the tumors following treatment. In certain embodiments, both the STING agonist and the cytokine are administered intratumorally to the patient.

In particular embodiments, the STING agonist employed in the methods, uses, and combination therapies disclosed herein is a CDN, such as a compound (“Compound A”) having the following structure, or a pharmaceutically acceptable salt thereof:

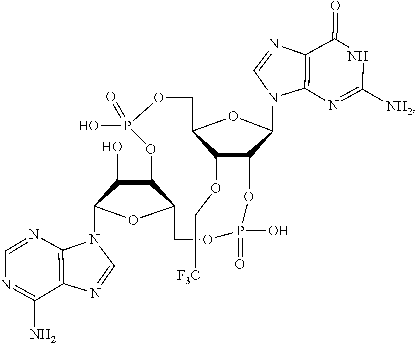

Compound A is a cyclic dinucleotide that is capable of activating STING and was described in U.S. Published Application No. 2018/0230177, which is incorporated herein by reference. Various salt forms of Compound A can be administered to a cancer patient. For instance, in one embodiment, an effective amount of a sodium salt of Compound A is administered to the cancer patient. It will be understood that any reference to Compound A in the disclosure also includes pharmaceutically acceptable salts thereof. In certain embodiments, Compound A is used in the methods, uses, and combination therapies disclosed here in combination with the cytokine IL-12.

In particular embodiments, the cytokine employed in the methods, uses, and combination therapies disclosed herein is an interleukin such as human interleukins IL-2, IL-7, IL-10, IL-12, IL-15, or a combination thereof. In certain embodiments, the interleukin is IL-2, IL-7, IL-10, IL-12, or a combination thereof. In some embodiments, the interleukin is IL-2, IL-12, IL-15, or a combination thereof. In one embodiment, the interleukin is IL-2. In another embodiment, the interleukin is IL-7. In another embodiment, the interleukin is IL-10. In another embodiment, the interleukin is IL-15. In a particular embodiment, the interleukin is IL-12. In certain other particular embodiments, the cytokine employed in the methods, uses, and combination therapies disclosed herein is an interleukin that is fused to a protein to form a fusion protein, such as IL-12 fused to collagen-binding lumican. In other particular embodiments, the cytokine employed in the methods, uses, and combination therapies disclosed herein is an interleukin that is fused to a protein to form a fusion protein, such as IL-2 fused to collagen-binding lumican. IL-12 or IL-2 fused to lumican are described in PCT publication WO 2020/068261, which is incorporated herein by reference. In certain embodiments, Compound A is used in the methods, uses, and combination therapies disclosed here in combination with the cytokine IL-12 fused to lumican.

### 5. BRIEF DESCRIPTION OF THE FIGURES

FIG. 1 shows the anti-tumor effect of a triple combination of a STING agonist (Compound A), an immune checkpoint inhibitor (anti-PD-L1 antibody), and a cytokine (IL-2, IL-12, or IL-15) in a mouse model. Panel A of FIG. 1 shows primary and distal tumor growth over time. Data is shown as mean±SEM. Panel B of FIG. 1 shows survival of the mice over time.

FIG. 2 shows the anti-tumor effect of a triple combination of a STING agonist (Compound A), an immune checkpoint inhibitor (anti-PD-L1 antibody), and a cytokine (IL-7 or IL-10) in a mouse model. Panel A of FIG. 2 shows primary and distal tumor growth over time. Data is shown as mean±SEM. Panel B of FIG. 2 shows survival of the mice over time.

FIGS. 3A, 3B, and 3C show the anti-tumor effect of a triple combination of a STING agonist (Compound A), an immune checkpoint inhibitor (anti-PD-L1 antibody), and various doses of IL-12 (50 ng for FIG. 3A, 200 ng for FIG. 3B, and 1 μg for FIG. 3C) in a mouse model. Panel A of each of FIGS. 3A, 3B, and 3C shows primary and distal tumor growth over time. Panel B of each of FIGS. 3A, 3B, and 3C shows survival of the mice over time. Panel C of each of FIGS. 3A, 3B, and 3C shows mouse body weight change over time. Data in panels A and C is shown as mean±SEM.

FIG. 4 shows the anti-tumor effect of a triple combination of a STING agonist (Compound A), an immune checkpoint inhibitor (anti-PD-L1 antibody), and various doses of IL-12 (3 ng, 10 ng, or 30 ng) in a mouse model. Panel A of FIG. 4 shows primary and distal tumor growth over time. Panel B of FIG. 4 shows survival of the mice over time. Panel C of FIG. 4 shows mouse body weight change over time. Data in panels A and C is shown as mean±SEM.

FIG. 5 shows the anti-tumor effect of combinations of a STING agonist (Compound A), an immune checkpoint inhibitor (anti-PD-L1 antibody), and IL-12-Fc in a mouse model. Panel A of FIG. 5 shows primary and distal tumor growth over time. Panel B of FIG. 5 shows survival of the mice over time. Panel C of FIG. 5 shows the body weight change over time. Data in panels A and C is shown as mean±SEM.

FIG. 6 shows the anti-tumor effect of a triple combination of a STING agonist (Compound A), an immune checkpoint inhibitor (anti-PD-L1 antibody), and various doses of IL-12-Fc (5 ng, 17 ng, or 50 ng) in a mouse model. Panel A of FIG. 6 shows primary and distal tumor growth over time. Panel B of FIG. 6 shows survival of the mice over time. Panel C of FIG. 6 shows the body weight change over time. Data in panels A and C is shown as mean±SEM.

FIG. 7 shows the anti-tumor effect of a triple combination of a STING agonist (Compound A), an immune checkpoint inhibitor (anti-PD-L1 antibody), and interleukins IL-12-Fc (30 ng) or IL12-MSA-Lumican (20 ng, 60 ng, or 200 ng) in a mouse model. Panel A of FIG. 7 shows primary and distal tumor growth over time. Panel B of FIG. 7 shows survival of the mice over time. Panel C of FIG. 7 shows mouse body weight change over time. Data in panels A and C is shown as mean±SEM.

FIG. 8 shows the anti-tumor effect of various combinations of a STING agonist (Compound A), an immune checkpoint inhibitor (anti-PD-L1 antibody), and interleukin IL12-MSA-Lumican in a mouse model. Panel A of FIG. 8 shows primary and distal tumor growth over time. Panel B of FIG. 8 shows survival of the mice over time. Data in panel A is shown as mean±SEM.

FIG. 9 shows the tumor growth in naïve mice or in mice previously treated with a triple combination of a STING agonist (Compound A), an immune checkpoint inhibitor (anti-PD-L1 antibody), and interleukin IL12-MSA-Lumican (20 ng, 60 ng, or 200 ng).

### 6. DETAILED DESCRIPTION

### 6.1. Definitions

As used in the specification and appended claims, unless specified to the contrary, the following terms and abbreviations have the meaning indicated:

“Combination therapy” refers herein to administration regimens of the recited substances for the particular recited administration routes for treating the recited disease. For example, a combination therapy for treating tumors in a cancer patient disclosed herein comprising a STING agonist and a cytokine, wherein both the STING agonist and the cytokine are formulated for intratumoral administration to the patient, would comprise intratumoral administration regimens for each of the STING agonist and the cytokine in sufficient dosing and frequency to treat tumors in the cancer patient.

“Conjointly administering” refers herein to any form of administration of two or more different therapeutic compounds such that the second administered compound is administered while the first administered therapeutic compound is still effective in the body (e.g., the two compounds are simultaneously effective in the patient, which may include additive or synergistic effects of the two compounds). For example, a STING agonist and a cytokine as disclosed herein can be administered either in the same formulation or in a separate formulation, either concomitantly or sequentially. In certain embodiments, the STING agonist and the cytokine disclosed herein can be administered within 1 hour, 2 hours, 12 hours, 24 hours, 36 hours, 48 hours, 72 hours, or a week of one another. In some embodiments, the STING agonist is administered first, and in other embodiments the cytokine is administered first. Thus, an individual who receives such treatment can benefit from a combined effect of the different therapeutic compounds.

“Effective amount” as used herein refers to an amount of the stated substance (e.g., a STING agonist, cytokine, or immune checkpoint inhibitor as disclosed herein) that is sufficient, when combined with another stated substance (e.g., a STING agonist, cytokine, or immune checkpoint inhibitor as disclosed herein) to treat the stated disease, disorder, or condition or have the desired stated effect on the disease, disorder, or condition or on one or more mechanisms underlying the disease, disorder, or condition or have the desired stated biological effect (e.g., augmenting anti-tumor response, increasing the population or function of immune cells, or increasing proliferation or function of tumor infiltrating leukocytes) in a human subject, such as a cancer patient. In certain embodiments, when a STING agonist is administered conjointly with a cytokine (and preferably but optionally with an immune checkpoint inhibitor) for the treatment of tumors, effective amounts refers to both an amount of the STING agonist and an amount of the cytokine (and an amount of the checkpoint inhibitor) which, upon conjoint administration to a human, treats, or ameliorates tumors in the human, or exhibits a detectable therapeutic or biological effect in the human. The therapeutic effect can be detected by, for example, a reduction in the size of one or more tumors, reduction in the proliferation of tumors, and increased survival times. The biological effect can be assessed by measuring the numbers of tumor infiltrating leukocytes using their surface markers such as CD45, determining the populations of specific immune cells including but not limited to T cells, NK cells, B cells, dendritic cells, or macrophages in tumor biopsies and in the blood, as well as measuring gene expression in single cells as well as in bulk cells in the tumors. The biological effect and safety of the therapies can also be examined by measuring a variety of inflammatory cytokines in the tumors and in the blood, by body weight and body temperature measurements, and by standard clinical and anatomical assessments as deemed necessary and appropriate by licensed clinicians.

“Reducing recurrence of tumors” or “preventing recurrence of tumors” in a cancer patient as used herein refers to reducing or preventing the recurrence of tumors in a cancer patient, who has been administered the specified agents (e.g., STING agonist, cytokine, and preferably with the optional immune checkpoint inhibitor), relative to a similarly afflicted cancer patient or patient type, who has not been administered the specified agents. In certain preferred embodiments, the reduction or prevention in recurrence of tumors occurs even when the patient does not receive further treatment with the specified agents. Without wishing to be bound by theory, in some instances, treatment with the specified agents, in addition to treating existing cancer/tumors, augments the anti-tumor response of the patient's immune system so as to reduce or prevent recurrence of tumors in the future after treatment with the specified agents has ended.

“Treatment” or “treating” as used herein refers to therapeutic applications associated with conjointly administering a STING agonist and a cytokine (and preferably but optionally with an immune checkpoint inhibitor) as disclosed herein that ameliorate the indicated disease, disorder, or condition or one or more underlying mechanisms of said disease, disorder, or condition, including slowing or stopping progression of the disease, disorder, or condition or one or more of the underlying mechanisms in a human subject, such as a cancer patient. In certain embodiments, when a STING agonist and a cytokine (and preferably but optionally with an immune checkpoint inhibitor) as disclosed herein are conjointly administered for the treatment of a treating tumors (such as in treating cancer), treatment refers to therapeutic applications to slow or stop progression of the tumors or the cancer and/or reversal of the tumors or the cancer. Reversal of tumors or the cancer differs from a therapeutic application that slows or stops tumors or the cancer in that with a method of reversing, not only is progression of the tumors or the cancer stopped, cellular behavior is moved to some degree toward a normal state that would be observed in the absence of the tumors or the cancer.

### 6.2. Administration of STING Agonists in Combination with Cytokines and Associated Combination Therapies

The disclosure provides methods of treating a disease or disorder, particularly cancer, in a patient in need thereof, such as a method of treating tumors in a cancer patient in need thereof, comprising administering in combination (e.g., conjointly) effective amounts of a STING agonist and a cytokine to the patient, wherein the STING agonist or the cytokine is intratumorally administered to the patient. In certain embodiments, the patient is currently receiving an immune checkpoint inhibitor as part of anti-tumor therapy. Conjoint administration contemplates that the STING agonist can be administered simultaneously, prior to, or after administration of the cytokine.

In a particular aspect, the disclosure provides a method of treating tumors in a cancer patient in need thereof, comprising administering in combination (e.g., conjointly) effective amounts of a STING agonist and a cytokine to the patient, wherein the STING agonist or the cytokine is intratumorally administered to the patient and wherein the patient exhibits reduced recurrence of the tumors following treatment. In certain embodiments, the patient is currently receiving an immune checkpoint inhibitor as part of anti-tumor therapy. Conjoint administration contemplates that the STING agonist can be administered simultaneously, prior to, or after administration of the cytokine.

In a further aspect, the disclosure provides a method of reducing recurrence of tumors in a cancer patient in need thereof, comprising administering in combination (e.g., conjointly) effective amounts of a STING agonist and a cytokine to the patient, wherein the STING agonist or the cytokine is intratumorally administered to the patient. In certain embodiments, the patient is currently receiving an immune checkpoint inhibitor as part of anti-tumor therapy. Conjoint administration contemplates that the STING agonist can be administered simultaneously, prior to, or after administration of the cytokine.

In another aspect, the disclosure provides a method of augmenting the anti-tumor response of a cancer patient, comprising administering in combination (e.g., conjointly) effective amounts of a STING agonist and a cytokine to the patient, wherein the STING agonist or the cytokine is intratumorally administered to the patient. In certain embodiments, the patient is currently receiving an immune checkpoint inhibitor as part of anti-tumor therapy. As discussed herein, the augmented anti-tumor response can be shown, for example, by shrinkage of one or more tumors or by increased survival times.

In yet another aspect, the disclosure provides a method of increasing the population or function of immune cells (such as T cells, NK cells, B cells, dendritic cells, or macrophages, or a combination thereof) of a cancer patient, comprising administering in combination (e.g., conjointly) effective amounts of a STING agonist and a cytokine to the patient, wherein the STING agonist or the cytokine is intratumorally administered to the patient. In certain embodiments, the patient is currently receiving an immune checkpoint inhibitor as part of anti-tumor therapy. In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages. As discussed herein, the increased population or function of immune cells can be shown, for example, by determining the populations of specific immune cells including but not limiting to T cells, NK cells, B cells, dendritic cells, or macrophages, or a combination thereof in tumor biopsies and in the blood.

In yet a further aspect, the disclosure provides a method of increasing proliferation or function of tumor infiltrating leukocytes in a cancer patient, comprising administering in combination (e.g., conjointly) effective amounts of a STING agonist and a cytokine to the patient, wherein the STING agonist or the cytokine is intratumorally administered to the patient.

In certain embodiments, the patient is currently receiving an immune checkpoint inhibitor as part of anti-tumor therapy. The increased proliferation or function of tumor infiltrating leukocytes can be shown, for example, by measuring the numbers of tumor infiltrating leukocytes using their surface markers such as CD45.

In some embodiments of the disclosed methods and uses, the STING agonist and the cytokine can both be administered intratumorally to a patient. In these embodiments, the STING agonist and the cytokine can be administered together in the same pharmaceutical composition or in separate pharmaceutical compositions. In other embodiments, the cytokine can be administered intratumorally to the patient, and the STING agonist can be administered systemically (e.g., intravenously, intramuscularly, subcutaneously, or orally) to the patient. In particular embodiments, the cytokine can be administered intratumorally to the patient, and the STING agonist can be administered intravenously to the patient. In particular embodiments, the cytokine can be administered intratumorally to the patient, and the STING agonist can be administered intramuscularly to the patient. In other embodiments, the cytokine can be administered intratumorally to the patient, and the STING agonist can be administered orally to the patient. In other embodiments, the STING agonist can be administered intratumorally to the patient, and the cytokine can be administered systemically (e.g., intravenously, intramuscularly, or subcutaneously) to the patient. In particular embodiments, the STING agonist can be administered intratumorally to the patient, and the cytokine can be administered intravenously to the patient. In particular embodiments, the STING agonist can be administered intratumorally to the patient, and the cytokine can be administered intramuscularly to the patient. In particular embodiments, the STING agonist can be administered intratumorally to the patient, and the cytokine can be administered subcutaneously to the patient. In certain embodiments, the method is a method of treating tumors in a cancer patient in need thereof. In certain embodiments, the method is a method of reducing recurrence of tumors in a cancer patient in need thereof. In certain embodiments, the method is a method of preventing recurrence of tumors in a cancer patient in need thereof. In certain embodiments, the method is a method of augmenting the anti-tumor response of a cancer patient. In certain embodiments, the method is a method of increasing the population or function of immune cells (such as T cells, NK cells, B cells, dendritic cells, or macrophages, or a combination thereof) of a cancer patient.

In embodiments where the STING agonist and cytokine are administered in separate compositions, the two compositions can be administered concomitantly or sequentially. In particular embodiments where the cytokine and the STING agonist are administered sequentially, the STING agonist can be administered prior to the administration of the cytokine. Alternatively, the STING agonist can be administered after administration of the cytokine.

In some embodiments, the STING agonist and the cytokine can be administered in combination, e.g., conjointly, without any additional therapeutic agents. Surprisingly, for some tumors, such as those exemplified herein, the combination of STING agonist and cytokine provides sufficient tumor inhibition such that additional chemotherapeutic agents or immunotherapeutic agents may not provide additional tumor inhibition.

However, in other embodiments, the STING agonist and the cytokine are administered in combination with one or more additional anti-cancer agents, such as in combination with an immune checkpoint inhibitor, such as a PD-1 inhibitor, a PD-L1 inhibitor, or a CTLA-4 inhibitor, including an anti-PD-1 antibody, an anti-PD-L1 antibody, and an anti-CTLA-4 antibody. In certain embodiments, the immune checkpoint inhibitor is an anti-PD-1 antibody. In certain embodiments, the immune checkpoint inhibitor is an anti-PD-L1 antibody. In certain embodiments, the immune checkpoint inhibitor is an anti-CTLA-4 antibody. Accordingly, in some embodiments, the disclosure provides a method of treating tumors in a cancer patient in need thereof, comprising administering in combination (e.g., conjointly) effective amounts of a STING agonist and a cytokine to the patient, wherein the STING agonist or the cytokine is intratumorally administered to the patient, and further comprising administering in combination (e.g., conjointly) an effective amount of an immune checkpoint inhibitor to the patient. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered. In particular embodiments, the immune checkpoint inhibitor is intramuscularly administered. In particular embodiments, the immune checkpoint inhibitor is subcutaneously administered. In particular embodiments, the patient is receiving an immune checkpoint inhibitor as part of anti-tumor therapy.

In a particular aspect, the disclosure provides a method of treating tumors in a cancer patient in need thereof, comprising administering in combination (e.g., conjointly) effective amounts of a STING agonist and a cytokine to the patient, wherein the STING agonist or the cytokine is intratumorally administered to the patient, and further comprising administering in combination (e.g., conjointly) an effective amount of an immune checkpoint inhibitor to the patient and wherein the patient exhibits reduced recurrence of the tumors following treatment. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered. In particular embodiments, the immune checkpoint inhibitor is intramuscularly administered. In particular embodiments, the immune checkpoint inhibitor is subcutaneously administered. In particular embodiments, the patient is receiving an immune checkpoint inhibitor as part of anti-tumor therapy.

In some embodiments, the methods and uses described herein comprise conjointly administering effective amounts of a STING agonist, a cytokine, and an immune checkpoint inhibitor to the patient, wherein the STING agonist and the cytokine are intratumorally administered to the patient and the immune checkpoint inhibitor is systematically administered to the patient and the immune checkpoint inhibitor is an anti-PD-1 antibody, an anti-PD-L1 antibody, or an anti-CTLA-4 antibody. In certain embodiments, the method is a method of treating tumors in a cancer patient in need thereof. In certain embodiments, the method is a method of reducing recurrence of tumors in a cancer patient in need thereof. In certain embodiments, the method is a method of preventing recurrence of tumors in a cancer patient in need thereof. In certain embodiments, the method is a method of augmenting the anti-tumor response of a cancer patient. In certain embodiments, the method is a method of increasing the population or function of immune cells (such as T cells, NK cells, B cells, dendritic cells, or macrophages, or a combination thereof) of a cancer patient.

In some embodiments, the methods and uses described herein comprise conjointly administering effective amounts of a STING agonist, a cytokine, and an immune checkpoint inhibitor to the patient, wherein the STING agonist and the cytokine are intratumorally administered to the patient and the immune checkpoint inhibitor is systematically administered to the patient and the immune checkpoint inhibitor is an anti-PD-1 antibody, an anti-PD-L1 antibody, or an anti-CTLA-4 antibody and wherein the STING agonist is a cyclic dinucleotide (CDN). In certain embodiments, the immune checkpoint inhibitor is an anti-PD-1 antibody. In certain embodiments, the immune checkpoint inhibitor is an anti-PD-L1 antibody. In certain embodiments, the immune checkpoint inhibitor is an anti-CTLA-4 antibody. In certain embodiments, the method is a method of treating tumors in a cancer patient in need thereof. In certain embodiments, the method is a method of reducing recurrence of tumors in a cancer patient in need thereof. In certain embodiments, the method is a method of preventing recurrence of tumors in a cancer patient in need thereof. In certain embodiments, the method is a method of augmenting the anti-tumor response of a cancer patient. In certain embodiments, the method is a method of increasing the population or function of immune cells (such as T cells, NK cells, B cells, dendritic cells, or macrophages, or a combination thereof) of a cancer patient.

In some embodiments, the methods and uses described herein comprise conjointly administering effective amounts of a STING agonist, a cytokine, and an immune checkpoint inhibitor to the patient, wherein the STING agonist and the cytokine are intratumorally administered to the patient and the immune checkpoint inhibitor is systematically administered to the patient and the immune checkpoint inhibitor is an anti-PD-1 antibody, an anti-PD-L1 antibody, or an anti-CTLA-4 antibody, and wherein the cytokine is an interleukin, and wherein the STING agonist is a cyclic dinucleotide (CDN). In certain embodiments, the immune checkpoint inhibitor is an anti-PD-I antibody. In certain embodiments, the immune checkpoint inhibitor is an anti-PD-L1 antibody. In certain embodiments, the immune checkpoint inhibitor is an anti-CTLA-4 antibody. In certain embodiments, the method is a method of treating tumors in a cancer patient in need thereof. In certain of such embodiments, the interleukin is IL-12, such as a fusion protein of IL-12, such as IL-12-Fc or IL-12-MSA-lumican. In certain embodiments, the method is a method of reducing recurrence of tumors in a cancer patient in need thereof. In certain embodiments, the method is a method of preventing recurrence of tumors in a cancer patient in need thereof. In certain embodiments, the method is a method of augmenting the anti-tumor response of a cancer patient. In certain embodiments, the method is a method of increasing the population or function of immune cells (such as T cells, NK cells, B cells, dendritic cells, or macrophages, or a combination thereof) of a cancer patient.

In some embodiments, the disclosure provides a method of treating of tumors in a cancer patient in need thereof, comprising causing a STING agonist, a cytokine, and an immune checkpoint inhibitor to be concurrently present in the patient's body. In some embodiments, the method comprises administering an effective amount of the STING agonist to the patient, wherein the patient has already been administered the cytokine and the immune checkpoint inhibitor. In some embodiments, the method comprises administering an effective amount of the cytokine to the patient, wherein the patient has already been administered the STING agonist and the immune checkpoint inhibitor. In some embodiments, the method comprises administering an effective amount of the immune checkpoint inhibitor to the patient, wherein the patient has already been administered the STING agonist and the cytokine.

In some embodiments, the disclosure provides a method of reducing recurrence of tumors in a patient, comprising causing a STING agonist, a cytokine, and an immune checkpoint inhibitor to be concurrently present in the patient's body. In some embodiments, the method comprises administering an effective amount of the STING agonist to the patient, wherein the patient has already been administered the cytokine and the immune checkpoint inhibitor. In some embodiments, the method comprises administering an effective amount of the cytokine to the patient, wherein the patient has already been administered the STING agonist and the immune checkpoint inhibitor. In some embodiments, the method comprises administering an effective amount of the immune checkpoint inhibitor to the patient, wherein the patient has already been administered the STING agonist and the cytokine.

In some embodiments, the disclosure provides a method of preventing recurrence of tumors in a patient, comprising causing a STING agonist, a cytokine, and an immune checkpoint inhibitor to be concurrently present in the patient's body. In some embodiments, the method comprises administering an effective amount of the STING agonist to the patient, wherein the patient has already been administered the cytokine and the immune checkpoint inhibitor. In some embodiments, the method comprises administering an effective amount of the cytokine to the patient, wherein the patient has already been administered the STING agonist and the immune checkpoint inhibitor. In some embodiments, the method comprises administering an effective amount of the immune checkpoint inhibitor to the patient, wherein the patient has already been administered the STING agonist and the cytokine.

In particular embodiments, the disclosure provides a method of treating tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the STING agonist is a CDN and/or the cytokine is an interleukin.

In a further particular embodiments, the disclosure provides a method of treating tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient and wherein the patient exhibits reduced recurrence of the tumors following treatment. In certain of such particular embodiments, the STING agonist is a CDN. In certain of such particular embodiments the cytokine is an interleukin, such as IL-12, such as a fusion protein of IL-12, such as IL-12-Fc or IL-12-MSA-lumican. In certain of such particular embodiments, the STING agonist is a CDN, and the cytokine is an interleukin, such as IL-12, such as a fusion protein of IL-12, such as IL-12-Fc or IL-12-MSA-lumican. In a particular embodiment, the patient exhibits reduced recurrence of the tumors following treatment.

In particular embodiments, the disclosure provides a method of reducing recurrence of tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the STING agonist is a CDN. In certain of such particular embodiments the cytokine is an interleukin, such as IL-12, such as a fusion protein of IL-12, such as IL-12-Fc or IL-12-MSA-lumican. In certain of such particular embodiments, the STING agonist is a CDN, and the cytokine is an interleukin, such as IL-12, such as a fusion protein of IL-12, such as IL-12-Fc or IL-12-MSA-lumican. In a particular embodiment, the patient exhibits reduced recurrence of the tumors following treatment.

In a particular embodiment, the disclosure provides a method of treating tumors in a cancer patient in need thereof, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient.

In particular embodiments, the disclosure provides a method of treating tumors in a cancer patient in need thereof, comprising conjointly administering effective amounts of a STING agonist, a cytokine, and an immune checkpoint inhibitor to the patient, wherein the STING agonist and the cytokine are intratumorally administered to the cancer patient, and the immune checkpoint inhibitor is systemically administered to the patient; and the immune checkpoint inhibitor is an anti-PD-I antibody, an anti-PD-L1 antibody, or an anti-CTLA-4 antibody.

In a particular embodiment, the disclosure provides a method of treating tumors in a cancer patient in need thereof, comprising conjointly administering effective amounts of a STING agonist that is a CDN, a cytokine that is IL-12, and an immune checkpoint inhibitor that is an anti-PD-I antibody, an anti-PD-L1 antibody, or an anti-CTLA-4 antibody to the patient, wherein both the CDN and the IL-12 are administered intratumorally to the patient, and the immune checkpoint inhibitor is intravenously administered to the patient. In certain of such particular embodiments, the IL-12 is a fusion protein of IL-12, such as IL-12-Fc or IL-12-MSA-lumican. In a particular embodiment, the patient exhibits reduced recurrence of the tumors following treatment.

In a further particular embodiment, the disclosure provides a method of treating tumors in a cancer patient in need thereof, comprising conjointly administering effective amounts of a STING agonist that is Compound A, a cytokine, and an immune checkpoint inhibitor that is an anti-PD-1 antibody, an anti-PD-L1 antibody, or an anti-CTLA-4 antibody to the cancer patient, wherein both the Compound A and the cytokine are administered intratumorally to the patient, and the immune checkpoint inhibitor is intravenously administered to the patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein, such as lumican. In certain of such particular embodiments, the cytokine is fused to an immunoglobulin Fc domain. In a particular embodiment, the patient exhibits reduced recurrence of the tumors following treatment.

In a further particular embodiment, the disclosure provides a method of treating tumors in a cancer patient in need thereof, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient and wherein the patient exhibits reduced recurrence of the tumors following treatment. In certain of such particular embodiments, the IL-12 is a fusion protein of IL-12, such as IL-12-Fc or IL-12-MSA-lumican.

In other embodiments, the disclosure provides a method of augmenting the anti-tumor response in a cancer patient, comprising administering in combination (e.g., conjointly) effective amounts of a STING agonist and a cytokine to the patient, wherein the STING agonist or the cytokine is intratumorally administered to the patient, and further comprising administering in combination (e.g., conjointly) an effective amount of an immune checkpoint inhibitor to the patient. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered. In particular embodiments, the immune checkpoint inhibitor is intramuscularly administered. In particular embodiments, the immune checkpoint inhibitor is subcutaneously administered. In particular embodiments, the patient is receiving an immune checkpoint inhibitor as part of anti-tumor therapy.

In particular embodiments, the disclosure provides a method of augmenting the anti-tumor response in a cancer patient, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the STING agonist is a CDN and/or the cytokine is an interleukin.

In a particular embodiment, the disclosure provides a method of augmenting the anti-tumor response in a cancer patient, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient.

In particular embodiments, the disclosure provides a method of augmenting the anti-tumor response in a cancer patient, comprising conjointly administering effective amounts of a STING agonist, a cytokine, and an immune checkpoint inhibitor to the patient, wherein the STING agonist and the cytokine are intratumorally administered to the patient, and the immune checkpoint inhibitor is systemically administered to the patient; and the immune checkpoint inhibitor is an anti-PD-1 antibody, an anti-PD-L1 antibody, or an anti-CTLA-4 antibody.

In a particular embodiment, the disclosure provides a method of augmenting the anti-tumor response in a cancer patient, comprising conjointly administering effective amounts of a STING agonist that is a CDN, a cytokine that is IL-12, and an immune checkpoint inhibitor that is an anti-PD-1 antibody, an anti-PD-L1 antibody, or an anti-CTLA-4 antibody to the patient, wherein both the CDN and the IL-12 are administered intratumorally to the patient, and the immune checkpoint inhibitor is intravenously administered to the patient. In certain of such particular embodiments, the IL-12 is a fusion protein of IL-12, such as IL-12-Fc or IL-12-MSA-lumican.

In a further particular embodiment, the disclosure provides a method of augmenting the anti-tumor response in a cancer patient, comprising conjointly administering effective amounts of a STING agonist that is Compound A, a cytokine, and an immune checkpoint inhibitor that is an anti-PD-1 antibody, an anti-PD-L1 antibody, or an anti-CTLA-4 antibody to the cancer patient, wherein both the Compound A and the cytokine are administered intratumorally to the patient, and the immune checkpoint inhibitor is intravenously administered to the patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In other embodiments, the disclosure provides a method of increasing the population or function of immune cells (such as T cells, NK cells, B cells, dendritic cells, or macrophages, or a combination thereof) in a cancer patient, comprising administering in combination (e.g., conjointly) effective amounts of a STING agonist and a cytokine to the patient, wherein the STING agonist or the cytokine is intratumorally administered to the patient, and further comprising administering in combination (e.g., conjointly) an effective amount of an immune checkpoint inhibitor to the patient. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered. In particular embodiments, the immune checkpoint inhibitor is intramuscularly administered. In particular embodiments, the immune checkpoint inhibitor is subcutaneously administered. In particular embodiments, the patient is receiving an immune checkpoint inhibitor as part of anti-tumor therapy. In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In particular embodiments, the disclosure provides a method of increasing the population or function of immune cells (such as T cells, NK cells, B cells, dendritic cells, or macrophages, or a combination thereof) in a cancer patient, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-I antibody, an anti-PD-L1 antibody, or an anti-CTLA-4 antibody to the cancer patient. In certain of such particular embodiments, the STING agonist is a CDN and/or the cytokine is an interleukin. In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In a particular embodiment, the disclosure provides a method of increasing the population or function of immune cells (such as T cells, NK cells, B cells, dendritic cells, or macrophages, or a combination thereof) in a cancer patient, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-I antibody, an anti-PD-L1 antibody, or an anti-CTLA-4 antibody to the cancer patient. In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In particular embodiments, the disclosure provides a method of increasing the population or function of immune cells (such as T cells, NK cells, B cells, dendritic cells, or macrophages, or a combination thereof) in a cancer patient, comprising conjointly administering effective amounts of a STING agonist, a cytokine, and an immune checkpoint inhibitor to the patient, wherein the STING agonist and the cytokine are intratumorally administered to the patient, and the immune checkpoint inhibitor is systemically administered to the patient; and the immune checkpoint inhibitor is an anti-PD-1 antibody, an anti-PD-L1 antibody, or an anti-CTLA-4 antibody. In certain embodiments, such methods increase the population or function of T cells.

In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In a particular embodiment, the disclosure provides a method of augmenting the anti-tumor response in a cancer patient, comprising conjointly administering effective amounts of a STING agonist that is a CDN, a cytokine that is IL-12, and an immune checkpoint inhibitor that is an anti-PD-1 antibody, an anti-PD-L1 antibody, or an anti-CTLA-4 antibody to the patient, wherein both the CDN and the IL-12 are administered intratumorally to the patient, and the immune checkpoint inhibitor is intravenously administered to the cancer patient. In certain of such particular embodiments, the IL-12 is a fusion protein of IL-12, such as IL-12-Fc or IL-12-MSA-lumican. In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In a further particular embodiment, the disclosure provides a method of augmenting the anti-tumor response in a cancer patient, comprising conjointly administering effective amounts of a STING agonist that is Compound A, a cytokine, and an immune checkpoint inhibitor that is an anti-PD-1 antibody, an anti-PD-L1 antibody, or an anti-CTLA-4 antibody to the cancer patient, wherein both the Compound A and the cytokine are administered intratumorally to the cancer patient, and the immune checkpoint inhibitor is intravenously administered to the patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In certain embodiments, such methods increase the population or function of T cells. In other embodiments, such methods increase the population or function of NK cells. In other embodiments, such methods increase the population or function of B cells. In other embodiments, such methods increase the population or function of dendritic cells. In other embodiments, such methods increase the population or function of macrophages.

In other embodiments, the disclosure provides a method of increasing proliferation or function of tumor infiltrating leukocytes in a cancer patient, comprising conjointly administering in combination (e.g., conjointly) a STING agonist and a cytokine to the patient, wherein the STING agonist or the cytokine is intratumorally administered to the patient, and further comprising administering in combination (e.g., conjointly) an effective amount of an immune checkpoint inhibitor to the patient. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered.

In particular embodiments, the disclosure provides a method of increasing proliferation or function of tumor infiltrating leukocytes in a cancer patient, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the STING agonist is a CDN and/or the cytokine is an interleukin. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In a particular embodiment, the disclosure provides a method of increasing proliferation or function of tumor infiltrating leukocytes in a cancer patient, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In one aspect, the disclosure provides methods of treating or preventing metastasis in a human cancer patient comprising administering in combination (e.g., conjointly) effective amounts of a STING agonist and a cytokine to the patient, wherein the STING agonist or the cytokine is intratumorally administered to the patient, and optionally further comprising administering in combination (e.g., conjointly) an effective amount of an immune checkpoint inhibitor to the patient. For instance, the methods can be used to treat primary or metastasizing tumors that are resistant to immune checkpoint therapy. In some such embodiments, the STING agonist and the cytokine are conjointly administered with a PD-1, PD-L1, or CTLA-4 inhibitor, or the cancer patient is currently receiving an immune checkpoint inhibitor as part of anti-tumor therapy. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In other embodiments, the disclosure provides a method of reducing recurrence of tumors in a cancer patient in need thereof, comprising administering in combination (e.g., conjointly) effective amounts of a STING agonist and a cytokine to the patient, wherein the STING agonist or the cytokine is intratumorally administered to the patient, and further comprising administering in combination (e.g., conjointly) an effective amount of an immune checkpoint inhibitor to the patient. In certain embodiments, the immune checkpoint inhibitor is intratumorally administered to the patient. In other embodiments, the immune checkpoint inhibitor is administered systemically (e.g., intravenously, intramuscularly, or subcutaneously) to the patient. In particular embodiments, the immune checkpoint inhibitor is intravenously administered. In particular embodiments, the immune checkpoint inhibitor is intramuscularly administered. In particular embodiments, the immune checkpoint inhibitor is subcutaneously administered. In particular embodiments, the patient is receiving an immune checkpoint inhibitor as part of anti-tumor therapy.

In particular embodiments, the disclosure provides a method of reducing recurrence of tumors in a cancer patient in need thereof, comprising conjointly administering effective amounts of a STING agonist and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient. In certain of such particular embodiments, the STING agonist is a CDN. In certain of such particular embodiments the cytokine is an interleukin, such as IL-12, such as a fusion protein of IL-12, such as IL-12-Fc or IL-12-MSA-lumican. In certain of such particular embodiments, the STING agonist is a CDN and the cytokine is an interleukin, such as IL-12, such as a fusion protein of IL-12, such as IL-12-Fc or IL-12-MSA-lumican.

In a particular embodiment, the disclosure provides a method of reducing recurrence of tumors in a cancer patient in need thereof, comprising conjointly administering effective amounts of a STING agonist that is a CDN and a cytokine that is IL-12 to the patient, wherein both the STING agonist and the cytokine are administered intratumorally to the patient, and the method further comprises conjointly systemically (e.g., intravenously) administering an effective amount of an immune checkpoint inhibitor that is an anti-PD-1 antibody, an anti-PD-L1 antibody, or an anti-CTLA-4 antibody to the cancer patient.

In a particular embodiment, the disclosure provides a method of reducing recurrence of tumors in a cancer patient in need thereof, comprising conjointly administering effective amounts of a STING agonist that is a CDN and a cytokine to the patient, wherein both the STING agonist and the cytokine are administered intratumorally to the patient, and the method further comprises conjointly systemically (e.g., intravenously) administering an effective amount of an immune checkpoint inhibitor that is an anti-PD-I antibody, an anti-PD-L1 antibody, or an anti-CTLA-4 antibody to the cancer patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican. In certain of such particular embodiments, the cytokine is fused to an immunoglobulin Fc domain.

In a particular embodiment, the disclosure provides a method of reducing recurrence of tumors in a cancer patient in need thereof, comprising conjointly administering effective amounts of a STING agonist that is a CDN, a cytokine that is IL-12, and an immune checkpoint inhibitor that is an anti-PD-1 antibody, an anti-PD-L1 antibody, or an anti-CTLA-4 antibody to the patient, wherein both the CDN and the IL-12 are administered intratumorally to the patient, and the immune checkpoint inhibitor is intravenously administered to the patient. In certain of such particular embodiments, the IL-12 is a fusion protein of IL-12, such as IL-12-Fc or IL-12-MSA-lumican.

In particular embodiments, the disclosure provides a method of reducing recurrence of tumors in a cancer patient in need thereof, comprising conjointly administering effective amounts of a STING agonist, a cytokine, and an immune checkpoint inhibitor to the patient, wherein the STING agonist and the cytokine are intratumorally administered to the cancer patient, and the immune checkpoint inhibitor is systemically administered to the patient; and the immune checkpoint inhibitor is an anti-PD-1 antibody, an anti-PD-L1 antibody, or an anti-CTLA-4 antibody.

In a further particular embodiment, the disclosure provides a method of reducing recurrence of tumors in a cancer patient in need thereof, comprising conjointly administering effective amounts of a STING agonist that is Compound A, a cytokine, and an immune checkpoint inhibitor that is an anti-PD-1 antibody, an anti-PD-L1 antibody, or an anti-CTLA-4 antibody to the cancer patient, wherein both the Compound A and the cytokine are administered intratumorally to the patient, and the immune checkpoint inhibitor is intravenously administered to the patient. In certain of such particular embodiments, the cytokine is fused to a protein to form a fused protein, wherein the protein is an antibody or an antibody fragment. In certain of such particular embodiments, the cytokine is fused to a protein to form a fused protein, wherein the protein is not an antibody or an antibody fragment. In certain of such particular embodiments, the cytokine is fused to a collagen-binding protein such as lumican.

In some embodiments, the disclosure provides a mixture comprising a STING agonist, a cytokine, and an immune checkpoint inhibitor. In some embodiments the mixture further comprises human plasma.

In particular embodiments, the disclosure provides a mixture comprising a STING agonist that is a CDN, a cytokine, an immune checkpoint inhibitor that is an anti-PD-1 antibody, an anti-PD-L1 antibody, or an anti-CTLA-4 antibody, and human plasma. In further particular embodiments, the disclosure provides a mixture comprising a STING agonist that is a CDN, a cytokine that is an interleukin, an immune checkpoint inhibitor that is an anti-PD-1 antibody, an anti-PD-L1 antibody, or an anti-CTLA-4 antibody, and human plasma. In yet further particular embodiments, the disclosure provides a mixture comprising a STING agonist that is Compound A, a cytokine that is an interleukin (such as IL-12), an immune checkpoint inhibitor that is an anti-PD-1 antibody, an anti-PD-L1 antibody, or an anti-CTLA-4 antibody, and human plasma. In certain of such particular embodiments, the IL-12 is a fusion protein of IL-12, such as IL-12-Fc or IL-12-MSA-lumican

Accordingly, in some embodiments, the STING agonist and the cytokine can be administered to a cancer patient in combination, e.g., conjointly, with a PD-1, PD-L1, or CTLA-4 inhibitor, such as those described herein. In such cases, the PD-1, PD-L1, or CTLA-4 inhibitor can be administered simultaneously with, prior to or after administration of the STING agonist and/or the cytokine. In some embodiments, the PD-1, PD-L1, or CTLA-4 inhibitor can be administered intratumorally. In other embodiments, the PD-1, PD-L1, or CTLA-4 inhibitor can be administered systemically, such as intravenously, subcutaneously, or intramuscularly. In certain embodiments, both the STING agonist and the cytokine are administered intratumorally to the cancer patient, and the PD-1, PD-L1, or CTLA-4 inhibitor is administered systemically, such as intravenously, subcutaneously, or intramuscularly. In other embodiments, the cytokine is administered intratumorally to the cancer patient, and both the STING agonist and the PD-1, PD-L1, or CTLA-4 inhibitor are administered systemically, such as intravenously, subcutaneously, intramuscularly, or orally. In other embodiments, the STING agonist is administered intratumorally to the cancer patient, and both the cytokine and the PD-1, PD-L1, or CTLA-4 inhibitor are administered systemically, such as intravenously, subcutaneously, or intramuscularly. In certain embodiments, both the cytokine and the PD-1, PD-L1, or CTLA-4 inhibitor are administered intratumorally to the cancer patient, and the STING agonist is administered systemically, such as intravenously, subcutaneously, intramuscularly, or orally. In some embodiments, both the STING agonist and the PD-1, PD-L1, or CTLA-4 inhibitor are administered intratumorally to the cancer patient, and the cytokine is administered systemically, such as intravenously, subcutaneously, or intramuscularly. In other embodiments, the STING agonist, the cytokine, and the PD-1, PD-L1, or CTLA-4 inhibitor are all administered intratumorally to the cancer patient.

In particular embodiments of the disclosed methods, the STING agonist and the cytokine are administered in combination, e.g., conjointly, with a CTLA-4 inhibitor and either a PD-1 inhibitor or a PD-L1 inhibitor. In certain of such embodiments, the CTLA-4 inhibitor is an anti-CTLA-4 antibody that is either intratumorally or systemically administered, particularly intratumorally administered.

In certain embodiments, the methods and uses described herein, upon administration of the STING agonist and the cytokine and optionally the immune checkpoint inhibitor, produce an abscopal effect in tumors distal to the site of intratumoral administration of the STING agonist or the cytokine. For example, in some embodiments the method and uses herein treat tumors distal to the site of intratumoral administration of the STING agonist and/or the cytokine. In some embodiments the method and uses herein treat tumors distal to the site of intratumoral administration of the STING agonist. In some embodiments the method and uses herein treat tumors distal to the site of intratumoral administration of the STING agonist and/or the cytokine.

In one embodiment, the STING agonist and cytokine are administered to a cancer patient already receiving immune checkpoint inhibition therapy, such as for whom the tumor or cancer has stabilized. In particular embodiments, the cancer patient has undergone at least 1 or 2 cycles of immune checkpoint inhibitor therapy prior to administration of the STING agonist and the cytokine. For instance, the cancer patient may have undergone 2, 3, 4, 5, 6, 7, or 8 cycles of immune checkpoint inhibition therapy prior to administration of the STING agonist and the cytokine. In certain of these embodiments, the cancer patient continues to receive immune checkpoint inhibition therapy with successive cycles of the STING agonist and cytokine.

In some embodiments, the present disclosure provides a combination therapy, such as for treating tumors in a cancer patient in need thereof, comprising a STING agonist and a cytokine, wherein the STING agonist or the cytokine is formulated for intratumoral administration to the patient. In certain embodiments, both the STING agonist and the cytokine are formulated for intratumoral administration to the patient. In some embodiments, the cytokine is formulated for intratumoral administration, and the STING agonist is formulated for systemic administration to the patient, such as for intravenous, subcutaneous, intramuscular, or oral administration. In certain embodiments, the STING agonist is formulated for intravenous administration. In certain embodiments, the STING agonist is formulated for subcutaneous administration. In certain embodiments, the STING agonist is formulated for intramuscular administration. In certain embodiments, the STING agonist is formulated for oral administration.

In other embodiments, STING agonist is formulated for intratumoral administration, and the cytokine is formulated for systemic administration to the patient, such as for intravenous, subcutaneous, or intramuscular administration. In certain embodiments, the cytokine is formulated for intravenous administration. In certain embodiments, the cytokine is formulated for subcutaneous administration. In certain embodiments, the cytokine is formulated for intramuscular administration.

In particular embodiments, the disclosure provides a combination therapy, such as for treating tumors in a cancer patient in need thereof, wherein both the STING agonist and the cytokine are formulated for intratumoral administration to the patient, and the combination therapy further comprises an immune checkpoint inhibitor that is an anti-PD-1 antibody, an anti-PD-L1 antibody, or an anti-CTLA-4 antibody formulated for systemic administration to the cancer patient. In certain of such particular embodiments, the STING agonist is a CDN and/or the cytokine is an interleukin.

In a particular embodiment, the disclosure provides a combination therapy for treating tumors in a cancer patient in need thereof, wherein the STING agonist is a CDN and the cytokine is IL-12, and both the STING agonist and the cytokine are formulated for intratumoral administration to the patient, and the combination therapy further comprises an immune checkpoint inhibitor that is an anti-PD-1 antibody, an anti-PD-L1 antibody, or an anti-CTLA-4 antibody formulated for systemic administration to the cancer patient.

In particular embodiments, the combination therapies disclosed herein further comprise an immune checkpoint inhibitor. In particular embodiments, the combination therapies disclosed herein further comprise an immune checkpoint inhibitor, such as a PD-1, PD-L1, or CTLA-4 inhibitor (such as an anti-PD-1 antibody, an anti-PD-L1 antibody, or an anti-CTLA-4 antibody).

In particular embodiments, the immune checkpoint inhibitor is an anti-PD-1 antibody. In particular embodiments, the immune checkpoint inhibitor is an anti-PD-L1 antibody. In particular embodiments, the immune checkpoint inhibitor is an CTLA-4 antibody. In some embodiments, the immune checkpoint inhibitor is formulated for intratumoral administration to the cancer patient. In other embodiments, the immune checkpoint inhibitor is formulated for systemic administration to the cancer patient, such as for intravenous, subcutaneous, or intramuscular administration. In certain embodiments, the immune checkpoint inhibitor is formulated for intravenous administration. In certain embodiments, the immune checkpoint inhibitor is formulated for subcutaneous administration. In certain embodiments, the immune checkpoint inhibitor is formulated for intramuscular administration.

In a particular embodiment, the disclosure provides a combination therapy, such as for treating tumors in a cancer patient in need thereof, wherein the STING agonist or the cytokine are formulated for intratumoral administration to the patient, and the combination therapy further comprises an immune checkpoint inhibitor that is an anti-PD-1 antibody, an anti-PD-L1 antibody, or an anti-CTLA-4 antibody formulated for systemic administration to the cancer patient. In a particular embodiment, the disclosure provides a combination therapy, such as for treating tumors in a cancer patient in need thereof, wherein both the STING agonist and the cytokine are formulated for intratumoral administration to the patient, and the combination therapy further comprises an immune checkpoint inhibitor that is an anti-PD-1 antibody, an anti-PD-L1 antibody, or an anti-CTLA-4 antibody formulated for systemic administration to the cancer patient.

In particular embodiments, the present disclosure provides a combination therapy, such as for treating tumors in a cancer patient in need thereof, comprising a STING agonist, a cytokine, and an immune checkpoint inhibitor, wherein the STING agonist and the cytokine are formulated for intratumoral administration to the patient, and the immune checkpoint inhibitor is formulated for systemic administration to the patient; and the immune checkpoint inhibitor is an anti-PD-1 antibody, and anti-PD-L1 antibody, or an anti-CTLA-4 antibody.

In a particular embodiment, the disclosure provides a combination therapy for treating tumors in a cancer patient in need thereof, wherein the STING agonist is a CDN, the cytokine is an interleukin, and both the STING agonist and the cytokine are formulated for intratumoral administration to the patient, and the immune checkpoint inhibitor is an anti-PD-I antibody, an anti-PD-L1 antibody, or an anti-CTLA-4 antibody and is formulated for systemic administration to the cancer patient. In certain of such particular embodiments, the STING agonist is Compound A. In certain of such particular embodiments, the cytokine is an interleukin that is IL-12. In a particular embodiment, the disclosure provides a combination therapy for treating tumors in a cancer patient in need thereof, wherein the STING agonist is Compound A, the cytokine is IL-12, and both the STING agonist and the cytokine are formulated for intratumoral administration to the patient, and the immune checkpoint inhibitor is an anti-PD-1 antibody, an anti-PD-L1 antibody, or an anti-CTLA-4 antibody and is formulated for systemic administration to the cancer patient. In certain of such particular embodiments, the cytokine is fused to a protein to form a fusion protein. In certain of such particular embodiments, the cytokine is fused to a protein to form a fusion protein, wherein the protein is an antibody or an antibody fragment. In certain of such particular embodiments, the cytokine is fused to a protein to form a fusion protein, wherein the protein is not an antibody or an antibody fragment.

### 6.3. STING Agonists. Cytokines, and Immune Checkpoint Inhibitors

In certain embodiments, the STING agonist used in the methods, uses, combination therapies, and mixtures described herein is a cyclic dinucleotide (CDN) compound. For instance, the STING agonist can be a 2′3′-CDN, such as 2′3′-cGAMP, Compound A, Compound B, or Compound C, depicted below, particularly Compound A. In other embodiments, the STING agonist is a 3′3′-CDN, a 2′2′-CDN, or a 3′2′-CDN, such as 3′3′-cGAMP, 2′2′-cGAMP, or 3′2′-cGAMP. In some embodiments, the STING agonist is a CDN that is an analog of 2′3′-cGAMP (i.e., a 2′3′-CDN that includes a guanine nucleobase and an adenine nucleobase), such as Compound A and Compound B, particularly Compound A.

In some embodiments, the STING agonist is a benzophenone analog. In further embodiments, the STING agonist is a dimeric aminobenzimidazole.

Examples of STING agonists that can be used in accordance with the disclosure include ADU-S100 (MIW815), BMS-986301, CRD5500, CMA (10-carboxymethyl-9-acridanone), diABZT STING agonist-1 (e.g., CAS No.: 2138299-34-8), DMXAA (ASA404/vadimezan), E7766, GSK-532, GSK-3745417, MK-1454, MK-2118, SB-11285, SRCB-0074, TAK-676, TTI-10001, SR-717 and MSA-2.

In one embodiment, the CDN used in the methods and combination therapies in accordance with the disclosure is the following compound (“Compound A”), or a pharmaceutically acceptable salt thereof:

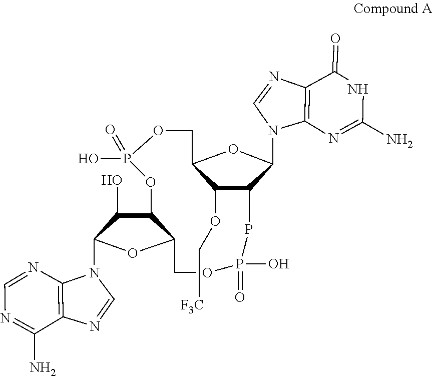

Compound A can act both locally and systemically to exert a powerful ant-tumor effect. Compound A, when administered at particular dosages to a cancer patient in need thereof, is capable of substantially reducing or preventing the spreading of metastasis. The ability of Compound A to reduce or prevent the onset and/or progression of metastasis can be potentiated when administered in combination, e.g., conjointly, with a cytokine, in accordance with the disclosure. Additionally, it has been discovered that Compound A exerts a powerful abscopal effect when administered in combination with a cytokine, in accordance with the present disclosure.

In some embodiments where Compound A serves as the STING agonist to be administered in combination with a cytokine, Compound A can be administered over multiple cycles. For instance, in one embodiment, the first cycle comprises administering Compound A on days 1, 8, and 15 of a four-week period, and subsequent cycles comprise administering Compound A on days 1 and 15 (i.e., biweekly) of a four-week period. Compound A can be administered intratumorally or systemically, including subcutaneously, intramuscularly, or intravenously. In some embodiments, on days of the cycle designated for administration, Compound A can be administered at a dosage in the range of 50 μg to 6,500 μg. In some embodiments, on days of the cycle designated for administration, Compound A can be administered at a dosage in the range of 100 μg to 3,000 μg. In some embodiments, on days of the cycle designated for administration, Compound A can be administered at a dosage in the range of 100 μg to 1,200 μg.

In one embodiment, the CDN used in the methods and combination therapies in accordance with the disclosure is the following compound (“Compound B”), or a pharmaceutically acceptable salt thereof:

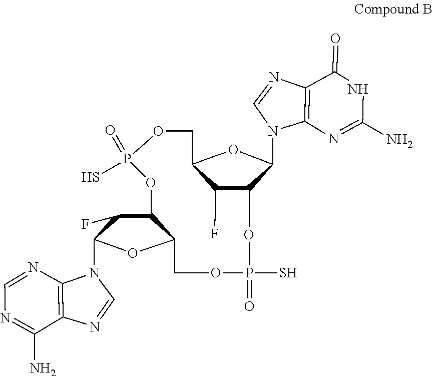

In another embodiment, the CDN used in the methods and combination therapies in accordance with the disclosure is the following compound (“Compound C”), or a pharmaceutically acceptable salt thereof:

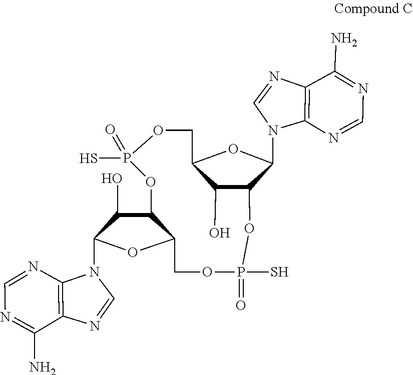

In another embodiment, the STING agonist used in the methods and combination therapies in accordance with the disclosure is a compound as disclosed in WO 2019/165032, which is herein incorporated by reference. Such STING agonists can be administered orally, systemically, or intratumorally to the patient. An example of one such STING agonist that can be used in accordance with the disclosure is SR-717 (“Compound D”), or a pharmaceutically acceptable salt thereof, which has the following structure:

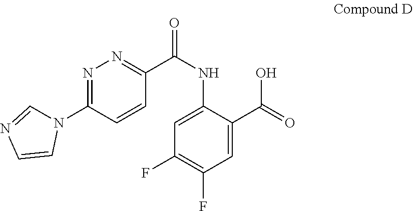 [SMILES: O=C(Nc1cc(F)c(F)cc1C(=O)O)c1ccc(-n2ccnc2)nn1]

In another embodiment, the STING agonist used in the methods and combination therapies in accordance with the disclosure is MSA-2 (“Compound E”), or a pharmaceutically acceptable salt thereof, which has the following structure:

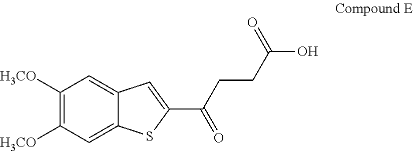 [SMILES: COc1cc2cc(C(=O)CCC(=O)O)sc2cc1OC]

MSA-2 can be administered orally, systemically, or intratumorally to the patient.

Additional examples of CDNs that can be used as STING agonists in the present methods and combination therapies are disclosed in the following publications WO 2014/144666, WO 2014/179335, WO 2014/189806, WO 2015/161762, WO 2016/096174, WO 2017/027646, WO 2017/027645, WO 2017/161349, WO 2018/118664, WO 2018/118665, WO 2018/208667, WO2019/165032, and WO 2019/046511 the contents of each of which are incorporated by reference herein.

In other embodiments, the STING agonist to be used in the methods and combination therapies in accordance with the disclosure can be conjugated to antibodies or antigen-binding fragments, hence producing antibody-drug conjugates (ADCs).

In one embodiment, the ADC to be administered in accordance with the methods and combination therapies disclosed herein has a structure as described in US 2017/0298139, WO 2017/100305, WO 2018/200812, or WO 2018/140831, the contents of each of which are herein incorporated by reference herein.

In particular embodiments, the ADC to be used in the methods and combination therapies in accordance with the disclosure has the structure of Formula IA:

Ab-[-L-D]ₙ (IA)

wherein:

“D” represents a CDN having the structure of Formula IIa:

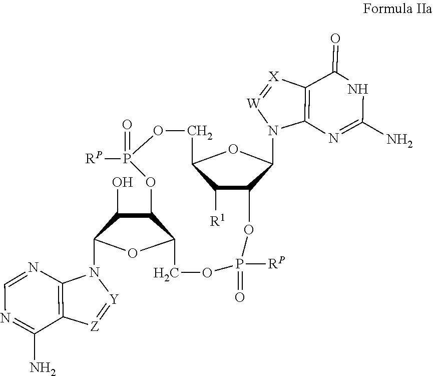

wherein
W, X, Y, and Z are independently CH or N;
R¹ is C₂₋₄alkyl substituted with a thiol, amino, or C₁₋₆alkylamino group;
R^{P} is, independently for each occurrence, hydroxyl, thiol, C₁₋₆alkyl, borano (—BH₃⁻), or —NR′R″, wherein R′ and R″ are, independently for each occurrence, hydrogen or C₁₋₆alkyl optionally substituted with one or more groups selected from halogen, thiol, hydroxyl, carboxyl, C₁₋₆alkoxy, C₁₋₆hydroxyalkoxy, —OC(O)C₁₋₆alkyl, —N(H)C(O)C₁₋₆alkyl, —N(C₁₋₃alkyl)C(O)C₁₋₆alkyl, amino, C₁₋₆alkylamino, di(C₁₋₆alkyl)amino, oxo, azido, and cyano; or R′ and R″ on the same nitrogen together form a C₃₋₅heterocyclic ring;
or a pharmaceutically acceptable salt thereof;
“Ab” represents an antibody or binding fragment thereof which binds a target antigen;
“L” represents, independently for each occurrence, a linker linking one or more occurrences of D to Ab;
“n” represents the number of occurrences of D linked to Ab via the linker (L);
wherein the CDN (D) is covalently bound to linker (L) at the thiol, amino, or C₁₋₆alkylamino group at the R′ position of the CDN.

In some embodiments wherein the STING agonist is part of an ADC of Formula IA, the CDN of the ADC has he structure of Formula IIb:

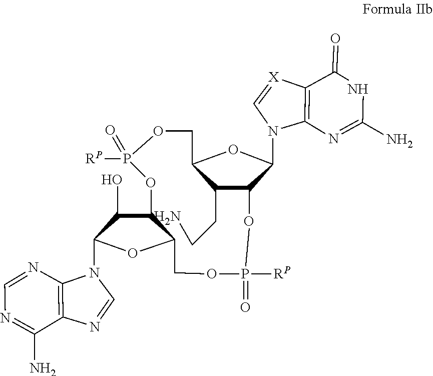

or a pharmaceutically acceptable salt thereof.

In some embodiments wherein the STING agonist is part of an ADC of Formula IA, the CDN of the ADC has he structure of Formula IIc:

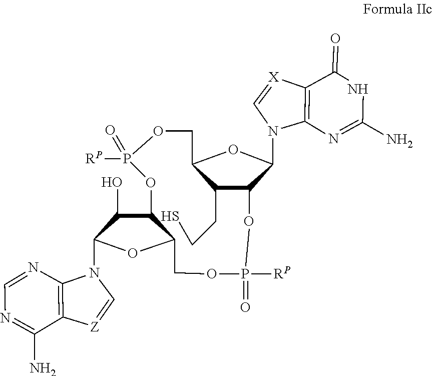

or a pharmaceutically acceptable salt thereof.

In some embodiments wherein the STING agonist is part of an ADC of Formula IA, the ADC has the structure of Formula III:

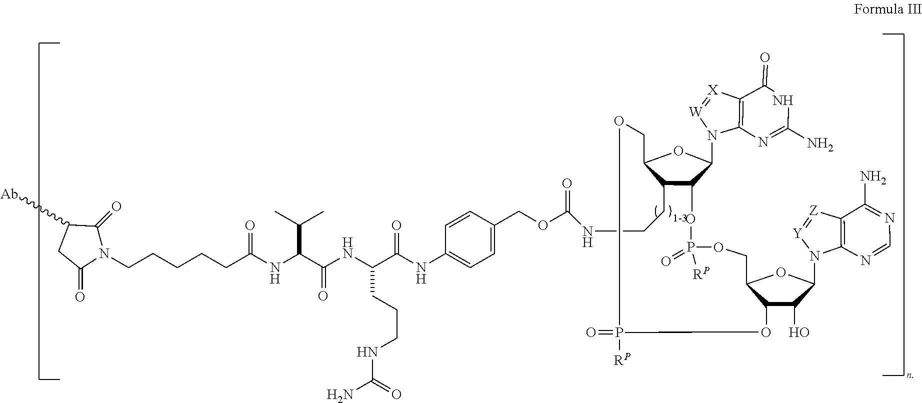

In some embodiments wherein the STING agonist is part of an ADC of Formula IA, the ADC has the structure of Formula IV:

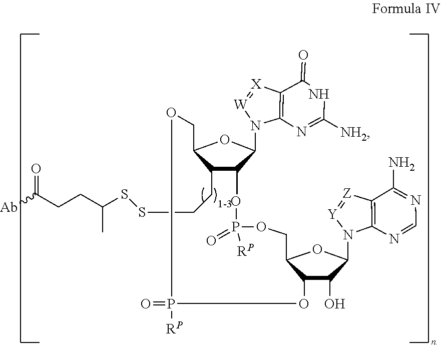

In some embodiments wherein the STING agonist is part of an ADC of Formula IA, the ADC (“Compound F”) has the following structure:

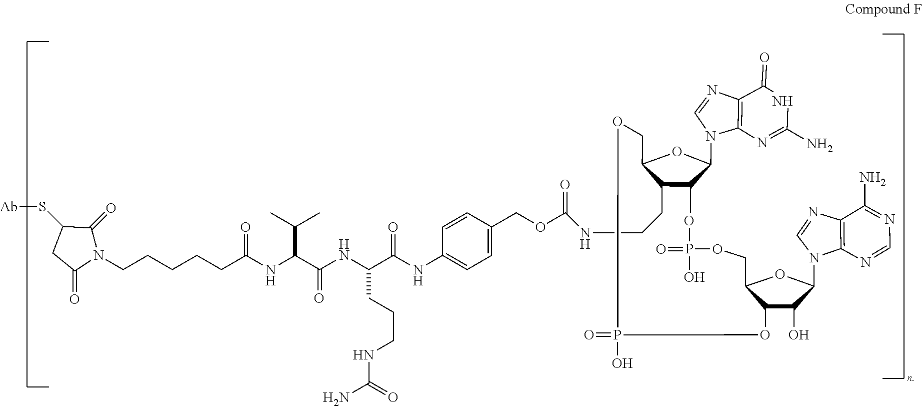

In some embodiments wherein the STING agonist is part of an ADC of Formula IA, the ADC (“Compound G”) has the following structure:

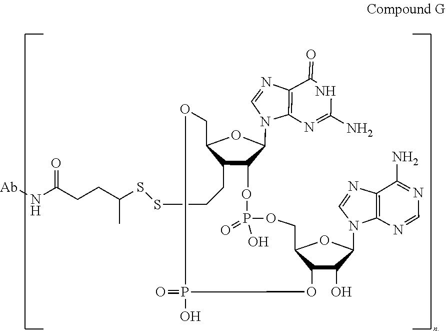

Examples of cytokines that can be used in the methods, uses, combination therapies, and mixtures disclosed herein include various interleukins, such as human interleukins IL-2, IL-7, IL-10, IL-12, IL-15, or a combination thereof. In certain embodiments, the interleukin is IL-2, IL-7, IL-10, IL-12, or a combination thereof. In some embodiments, the interleukin is IL-2, IL-12, IL-15, or a combination thereof. In one embodiment, the interleukin is IL-2. In another embodiment, the interleukin is IL-7. In another embodiment, the interleukin is IL-10. In another embodiment, the interleukin is IL-15. In a particular embodiment, the interleukin is IL-12.

In certain embodiments, the interleukin is fused to a protein to form a fusion protein, wherein the protein is an antibody or an antibody fragment. In certain embodiments the interleukin is fused to an antibody to form a fusion protein. In certain embodiments, the interleukin is fused to an antibody fragment to form a fusion protein. In certain embodiments the interleukin is fused to a protein to form a fusion protein, wherein the protein is not an antibody or an antibody fragment.

Examples of interleukin fusion proteins with lumican that can be used in the present methods, uses, and combination therapies are disclosed in PCT publication WO 2020/068261 the contents of each of which are incorporated by reference.

In certain embodiments, the interleukin is fused to a protein to form a fusion protein, wherein the protein is not an antibody or an antibody fragment. In particular embodiments, the interleukin is fused to a collagen-binding protein. In particular embodiments, the collagen-binding protein is lumican. In particular embodiments, the interleukin in the fusion protein is IL-12. In particular embodiments, the interleukin in the fusion protein is IL-2.

In certain embodiments, the interleukin is fused to a protein to form a fusion protein, wherein the protein is not an antibody or an antibody fragment. In particular embodiments, the interleukin is fused to an IL-2 receptor alpha chain, prostate-specific antigen cleavage sequence, matrix metalloproteinase cleavage sequence, or an alum-binding peptide. In particular embodiments, the interleukin in the fusion protein is IL-12. In particular embodiments, the interleukin in the fusion protein is IL-2.

In certain embodiments, the interleukin is IL-12 and is fused to a protein to form a fusion protein, wherein the protein is an antibody or an antibody fragment. In certain embodiments the interleukin is fused to an antibody to form a fusion protein. In certain embodiments, the interleukin is fused to an antibody fragment to form a fusion protein. In certain embodiments the interleukin is fused to a protein to form a fusion protein, wherein the protein is not an antibody or an antibody fragment. In certain embodiments, the interleukin is fused to an antibody that recognizes DNA/histone complexes. In certain embodiments, the interleukin is fused to the human monoclonal IgG1 antibody NHS76. Example of IL-12 fused to IgG1 antibody NHS76 is disclosed in Greiner et al., 2021, Immunotargets Ther. May 27; 10:155-169. In certain embodiments, the interleukin can be fused to an IL-2 receptor alpha chain, prostate-specific antigen cleavage sequence, matrix metalloproteinase cleavage sequence, or antibody fragment scFv. Examples of such interleukin fusion proteins are disclosed in Puskas et al., 2011, Immunology, June; 133(2):206-20. In certain embodiments, the interleukin can be fused to an alumn binding peptide (ABP). Example of an iterleukin bound to ABP is disclosed in Agarwal et al., 2022, Nat Biomed Eng 6, 129-143; and Puskas et al., 2011, Immunology, June; 133(2):206-20.

In certain embodiments, the interleukin is IL-2 and is fused to a protein to form a fusion protein, wherein the protein is an antibody or an antibody fragment. In certain embodiments the interleukin is fused to an antibody to form a fusion protein. In certain embodiments, the interleukin is fused to an antibody fragment to form a fusion protein. In certain embodiments the interleukin is fused to a protein to form a fusion protein, wherein the protein is not an antibody or an antibody fragment. In certain embodiments, the interleukin is a fusion protein,

In certain embodiments, the interleukin is a fusion protein, such as an Fc-fused interleukin, such as Fc-fused IL-2, IL-7, IL-10, IL-12, IL-15, or a combination thereof. In certain embodiments, the interleukin is Fc-fused IL-2, IL-7, IL-10, IL-12, or a combination thereof. In some embodiments, the interleukin is Fc-fused IL-2, IL-12, IL-15, or a combination thereof. In one embodiment, the interleukin is Fc-fused IL-2. In another embodiment, the interleukin is Fc-fused IL-7. In another embodiment, the interleukin is Fc-fused IL-10. In another embodiment, the interleukin is Fc-fused IL-15. In a particular embodiment, the interleukin is Fc-fused IL-12. In other embodiments, the interleukin is not a fusion protein.

As discussed above, the immune checkpoint inhibitor, when used in the methods and combination therapies disclosed herein, can be a PD-1 inhibitor, a PD-L1 inhibitor, or a CTLA-4 inhibitor, including an anti-PD-1 antibody, an anti-PD-L1 antibody, and an anti-CTLA-4 antibody

Examples of CTLA-4 inhibitors that can be used in accordance with the present disclosure include, but are not limited to, ipilimumab (Yervoy®) and tremelimumab (ticilimumab), CBT-509, CS1002, BMS-986249, AGEN1181, AGEN1194, AGN2041, BA3071, ATOR-1015, ATOR-1144, ADV-1604 and BCD-145. In particular embodiments, the CTLA-4 inhibitor is an anti-CTLA-4 antibody selected from ipilimumab (Yervoy®) and tremelimumab.

The CTLA-4 inhibitor can generally be administered systemically or intratumorally, and in particular embodiments the CTLA-4 inhibitor is an anti-CTLA-4 antibody that is administered intratumorally.

In some embodiments, when the immune checkpoint inhibitor is a CTLA-4 inhibitor, such as an anti-CTLA-4 antibody, the CTLA-4 inhibitor inhibits the interaction between CTLA-4 on T cells and CD80 (B7.1) or CD86 (B7.2) on an antigen presenting cell such as a dendritic cell or a macrophage in the tumor microenvironment.

Examples of PD-1 inhibitors that can be used in accordance with the present disclosure include, but are not limited to, pembrolizumab (Keytruda®), nivolumab (Opdivo®), cemiplimab (Libtayo®), AMP-224, AMP-514, or PDR001. The PD-1 inhibitor can generally be administered systemically or intratumorally.

Examples of PD-L1 inhibitors that can be used in accordance with the present disclosure include, but are not limited to, atezolizumab (Tecentriq®), avelumab (Bavencio®), durvalumab (Imfinzi®), BMS-936559, or CK-301. The PD-L1 inhibitor can generally be administered systemically or intratumorally.

### 6.4. Further Methods of Treatment

In some embodiments, both the STING agonist and the cytokine are administered intratumorally into the primary tumor of the patient. It has been found that when particular STING agonists (e.g., Compound A) are administered intratumorally into the primary tumor, tumor growth is suppressed not only at the site of the primary tumor, but also at the site of distant tumors. Therefore, such STING agonists display an abscopal effect. Moreover, the STING agonist can augment T cell priming and inflammation in the tumor microenvironment, at both the site of injection and at distal legions. Cytokines, such as the interleukins, can expand T cells. Accordingly, the present disclosure contemplates that the combination of a STING agonist and a cytokine results in increased, even synergistic, proliferation and/or function of T cells, which produces in an even larger abscopal effect than administering the STING agonist in the absence of a cytokine. However, the present disclosure provides such combination therapies involving a STING agonist and a cytokine while reducing or limiting systemic toxicity from the cytokine.

Accordingly, the disclosure provides methods of treating both primary and distant tumors (including accessible and inaccessible cancers) by administering the combination therapies disclosed herein. In certain embodiments, the methods described herein treat a tumor distant from the site of intratumoral administration of the STING agonist and/or the cytokine.

The present disclosure also provides a method of treating a patient, who is concurrently being treated systemically (e.g., intravenously, intramuscularly, subcutaneously, orally) or intratumorally with a STING agonist as described herein, comprising administering to the patient a cytokine as described herein. In certain embodiments, the cytokine is administered intratumorally. In other embodiments, the cytokine is administered systemically (e.g., intravenously, intramuscularly, or subcutaneously). In some embodiments, the method further comprises administering a PD-1 inhibitor (e.g., an anti-PD-1 antibody), a PD-L1 inhibitor (e.g., an anti-PD-L1 antibody), or a CTLA-4 inhibitor (e.g., an anti-CTLA-4 antibody) as described herein to the patient. In certain of these embodiments, the patient is suffering from a cancer, such as those described herein. In some embodiments, the method of treating the patient treats the patient for the cancer.

In particular embodiments, the combination therapies of the disclosure can be used to treat cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney, or liver. Further cancers treatable by the combination therapies of thee disclosure include rectal cancer; cancer of the anal region; carcinomas of the fallopian tubes, endometrium, cervix, vagina, vulva, renal pelvis, and renal cell; sarcoma of soft tissue; myxoma; rhabdomyoma, fibroma; lipoma; teratoma, cholangiocarcinoma; hepatoblastoma; angiosarcoma; hemangioma; hepatoma; fibrosarcoma; chondrosarcoma; myeloma; chronic or acute leukemia; lymphocytic lymphomas; primary CNS lymphoma; neoplasms of the CNS; spinal axis tumors; squamous cell carcinomas; synovial sarcoma; malignant pleural mesotheliomas; brain stem glioma; pituitary adenoma; bronchial adenoma; chondromatous hanlartoma; inesothelioma; Hodgkin's Disease; or a combination of one or more of the foregoing cancers.

In particular embodiments, the combination therapies of the disclosure can be used to treat a cancer that is refractory or unresponsive to immune checkpoint inhibitory therapy. In some instances, such cancers exhibit tumors of low immunogenicity. Such cancers may include but are not limited to prostate cancer, pancreatic cancer, lymphoma, head and neck cancer, kidney cancer, melanoma, colon cancer, breast cancer, and lung cancer. In certain embodiments, the cancer is selected from prostate cancer, pancreatic cancer, lymphoma, head and neck cancer, and kidney cancer. In some embodiments, the cancer is selected from melanoma, colon cancer, breast cancer, and lung cancer.

In certain embodiments, the combination therapies and methods of the disclosure are useful in treating solid tumors, such as tumors associated with melanoma or cancers of the kidney, lung, liver, colon, pancreas, brain, head and neck, bladder, prostate, breast, ovarian, cervix, and thyroid. In some instances, the present combination therapies and methods are useful in treating such tumors when they are the primary tumor.

In other embodiments, the combination therapies and methods of the disclosure are useful in treating metastatic cancers that are capable of or have already spread to multiple organs.

In particular embodiments, the combination therapies of the disclosure can be used to reduce the recurrence of the tumors following the initial treatment.

It will be appreciated by the skilled worker that methods disclosed herein are disclosed also as their corresponding “Swiss-type” or “EPC2000” equivalent. Thus a method of treating tumors in a cancer patient in need thereof, comprising conjointly administering effective amounts of a STING agonist and a cytokine to the patient, would be understood also to disclose the use of a STING agonist in the manufacture of a medicament for treating tumors in a cancer patient in need thereof, wherein the treating comprises conjointly administering effective amounts of the STING agonist and a cytokine to the patient, or the use of a cytokine in the manufacture of a medicament for treating tumors in a cancer patient in need thereof, wherein the treating comprises conjointly administering effective amounts of the cytokine and a STING agonist to the patient. Likewise, disclosure of the above method would be understood to disclose the combination of a STING agonist and a cytokine for treating tumors in a cancer patient in need thereof.

### 6.5. Pharmaceutical Compositions and Kits

The disclosure further provides for a pharmaceutical composition comprising a STING agonist, a cytokine, and a pharmaceutically acceptable carrier. In certain embodiments, the pharmaceutical composition is an injectable pharmaceutical composition, e.g., for intratumoral injection. In some embodiments, the pharmaceutical acceptable carrier may include physiological saline or phosphate buffered saline (PBS). A particular advantage provided by the disclosure is that the STING agonist and the cytokine can be administered intratumorally in a single composition. Administration of a single composition reduces the number of injections required and reduces incidence of side effects associated with administration of multiple doses of the individual therapeutic agents. Moreover, because of the synergy observed when the cytokine is administered together with the STING agonist, the dose of either of the agents to achieve efficacy is less than the dose to achieve efficacy when either of the agents is administered as a monotherapy. Accordingly, incidence of side effects such as irritation is further reduced by this synergy.

In other embodiments, the present disclosure provides a kit for treating a disease or disorder, including cancer, the kit comprising a STING agonist and a cytokine. In certain embodiments, the kit provides the cytokine formulated for intratumoral administration and the STING agonist formulated for intratumoral or systemic (e.g., intravenous, intramuscular, subcutaneous, or oral) administration. In other embodiments, the kit provides the STING agonist formulated for intratumoral administration and the cytokine formulated for intratumoral or systemic (e.g., intravenous, intramuscular, or subcutaneous) administration. In some embodiments, both the STING agonist and cytokine are formulated for intratumoral administration.

In certain embodiments, the kit further comprises a PD-1 inhibitor (e.g., an anti-PD-1 antibody), a PD-L1 inhibitor (e.g., an anti-PD-L1 antibody), or a CTLA-4 inhibitor (e.g., an anti-CTLA-4 antibody). In some of such embodiments, the PD-1 inhibitor, PD-L1 inhibitor, or CTLA-4 inhibitor are formulated for intratumoral or systemic (e.g., intravenous, intramuscular, or subcutaneous) administration. In certain embodiments, both the cytokine and STING agonist are formulated for intratumoral administration, and the PD-1 inhibitor, PD-L1 inhibitor, or CTLA-4 inhibitor is formulated for systemic (e.g., intravenous, intramuscular, or subcutaneous) administration. In other embodiments, the cytokine is formulated for intratumoral administration, and both the STING agonist and the PD-I inhibitor, PD-L1 inhibitor, or CTLA-4 inhibitor are formulated for systemic (e.g., intravenous, intramuscular, subcutaneous, or oral) administration. In certain embodiments, both the cytokine and the PD-1 inhibitor, PD-L1 inhibitor, or CTLA-4 inhibitor are formulated for intratumoral administration, and the STING agonist is formulated for systemic (e.g., intravenous, intramuscular, subcutaneous, or oral) administration. In some embodiments, both the STING agonist and the PD-1 inhibitor, PD-L1 inhibitor, or CTLA-4 inhibitor are formulated for intratumoral administration, and cytokine is formulated for systemic (e.g., intravenous, intramuscular, or subcutaneous) administration. In other embodiments, the STING agonist, the cytokine, and the PD-1 inhibitor, PD-L1 inhibitor, or CTLA-4 inhibitor are all formulated for intratumoral administration.

### 6.6. Dosing Regimens

The dosage of the STING agonist will vary depending on the particular STING agonist and the route of administration. In general, for systemic or intratumoral administration, the STING agonist can be administered at a dose in the range of 1-1000 μg/kg. For oral administration, the STING agonist can be administered at a dose in the range of 5-5000 μg/kg.

In particular embodiments, where the STING agonist is a 2′3′-cGAMP analog, such as Compound A, the STING agonist can be administered intratumorally or systemically in the range of 1-100 μg/kg. For instance, a 2′3′-cGAMP analog, such as Compound A, can be administered to a cancer patient in the range of 1-10 μg/kg, 5-10 μg/kg, 5-20 μg/kg, 5-30 μg/kg, 5-40 μg/kg, 5-50 μg/kg, 10-20 μg/kg, 10-30 μg/kg, 10-40 μg/kg, 10-50 μg/kg, 15-20 μg/kg, 15-40 μg/kg, 20-30 μg/kg, 20-40 μg/kg, 20-50 μg/kg, 30-40 μg/kg, 30-50 μg/kg, 5-75 μg/kg, 10-75 μg/kg, 15-75 μg/kg, 20-75 μg/kg, 25-75 μg/kg, 35-75 μg/kg, 5-100 μg/kg, 10-100 μg/kg, 15-100 μg/kg, 20-100 μg/kg, 25-100 μg/kg, 35-100 μg/kg, or 50-100 μg/kg.

In some embodiments, a 2′3′-cGAMP analog, such as Compound A, can be administered to a cancer patient at a dose, e.g., a single or divided doses, in the range of 10-6,500 μg, such as 50-6,500 μg. In particular embodiments, a 2′3′-cGAMP analog, such as Compound A, can be administered to a cancer patient at a dosage, e.g., a single or divided doses, in the range of 100-3,000 μg. In other embodiments, a 2′3′-cGAMP analog, such as Compound A, can be administered to a cancer patient at a dosage e.g., a single or divided doses, in the range of 100-1,200 μg. For instance, a 2′3′-cGAMP analog, such as Compound A, can be administered to a cancer patient in the range of 10-50 μg, 10-100 μg, 10-200 μg, 50-200 μg, 100-200 μg, 100-400 μg, 100-500 μg, 100-800 μg, 200-400 μg, 400-600 μg, 400-800 μg, 100-1,000 μg, 250-1,000 μg, 500-1,000 μg, 500-3,000 μg, 1,000-3,000 μg, 500-4,500 μg, 1,000-4,500 μg, 500-6,500 μg, 1,000-6,500 μg, 2,000-6,500 μg, 3,000-6,500 μg, or 4,500-6,500 μg.

In embodiments involving the administration of priming and maintenance doses of a 2′3′-cGAMP analog, such as Compound A, the priming dose of can be administered to a cancer patient at a dosage in the range of 10-1,000 μg. For instance, the priming dose of a 2′3′-cGAMP analog, such as Compound A, can be administered to a cancer patient in the range of 10-20 μg, 10-40 μg, 10-50 μg, 10-80 μg, 20-40 μg, 40-60 μg, 40-80 μg, 50-100 μg, 100-200 μg, 100-300 μg, 100-500 μg, 200-500 μg, 200-800 μg, 200-1,000 μg, 500-800 μg, or 500-1,000 μg. In certain embodiments, the priming dose of a 2′3′-cGAMP analog, such as Compound A, can be administered to a cancer patient at a dosage in the range of 0.15-20 μg/kg, such as 0.15-1 μg/kg, 0.25-1 μg/kg, 0.5-1 μg/kg, 0.5-2 μg/kg, 1-3 μg/kg, 1-5 μg/kg, 2-5 μg/kg, 2-7 μg/kg, 1-10 μg/kg, 2-10 μg/kg, 3-10 μg/kg, 5-10 μg/kg, 5-15 μg/kg, 10-20 μg/kg, or 15-20 μg/kg.

In embodiments involving the administration of priming and maintenance doses of a 2′3′-cGAMP analog, such as Compound A, the maintenance doses can be administered to a cancer patient at a dosage in the range of 100-3,000 μg. In other embodiments, the maintenance doses of a 2′3′-cGAMP analog, such as Compound A, can be administered to a cancer patient at a dosage in the range of 100-1,200 μg. For instance, the maintenance doses of a 2′3′-cGAMP analog, such as Compound A, can be administered to a cancer patient in the range of 50-200 μg, 100-200 μg, 100-400 μg, 100-500 μg, 100-800 μg, 100-1,000 μg, 200-400 μg, 200-800 μg, 200-1,200 μg, 250-1,000 μg, 400-600 μg, 400-800 μg, 400-1,200 μg, 500-1,000 μg, 500-1,200 μg, 500-1,500 μg, 500-2,000 μg, 500-4,500 μg, 800-1,200 μg, 800-1,500 μg, 800-2,000 μg 1,000-2,000 μg, 1,000-3,000 μg, 1,000-4,500 μg, 2,0004,500 μg, 500-6,500 μg, 1,000-6,500 μg, 1,500-6,500 μg, 2,000-6,500 μg, or 3,000-6,500 μg. In certain embodiments, the maintenance doses of a 2′3′-cGAMP analog, such as Compound A, can be administered to a cancer patient at a dosage in the range of 1-100 μg/kg, such as 1-50 μg/kg. For instance, the maintenance doses of a 2′3′-cGAMP analog, such as Compound A, can be administered to a cancer patient in the range of 1-10 μg/kg, 5-10 μg/kg, 5-20 μg/kg, 5-30 μg/kg, 5-40 μg/kg, 5-50 μg/kg, 10-20 μg/kg, 10-30 μg/kg, 10-40 μg/kg, 10-50 μg/kg, 15-20 μg/kg, 15-40 μg/kg, 20-30 μg/kg, 20-40 μg/kg, 20-50 μg/kg, 30-40 μg/kg, 30-50 μg/kg, 5-75 μg/kg, 10-75 μg/kg, 15-75 μg/kg, 20-75 μg/kg, 25-75 μg/kg, 35-75 μg/kg, 5-100 μg/kg, 10-100 μg/kg, 15-100 μg/kg, 20-100 μg/kg, 25-100 μg/kg, 35-100 μg/kg, or 50-100 μg/kg.

In another embodiment, the dosing cycle comprises administering a priming dose of a 2′3′-cGAMP analog, such as Compound A, on day 1 of a treatment cycle followed by administering a 2′3′-cGAMP analog, such as Compound A, under two maintenance dosing regimens. The first maintenance dosing regimen comprises administering maintenance doses of a 2′3′-cGAMP analog, such as Compound A, on days 8, 15 and 22 (i.e., the first day of weeks 2, 3 and 4) of the treatment cycle, followed by a period of one week (i.e., week 5) where the 2′3′-cGAMP analog is not administered to the patient. The second maintenance dosing regimen comprises administering a 2′3′-cGAMP analog, such as Compound A, on a biweekly dosing regimen. For instance, a 2′3′-cGAMP analog, such as Compound A, can be administered at the beginning of weeks 6 and 8 of the dosing cycle. In some embodiments, additional biweekly dosing of a 2′3′-cGAMP analog, such as Compound A, can be administered to the patient. For instance, a 2′3′-cGAMP analog, such as Compound A, can be administered at week 10 of the dosing cycle, weeks 10 and 12 of the dosing cycle, weeks 10, 12, and 14 of the dosing cycle, weeks 10, 12, 14, and 16 of the dosing cycle, and so on.

In general, for systemic or intratumoral administration, the amount of the cytokine, such as an interleukin, administered to a cancer patient can be in the range of 0.001 μg/kg to 2 mg/kg, particularly 0.01 μg/kg to 1 mg/kg, depending on the cytokine or interleukin used.

For example, for IL-12, the amount of the cytokine intratumorally administered to a cancer patient can be in the range of 0.01-100 μg/kg, such as in the range of 0.01-0.1 μg/kg, 0.01-1 μg/kg, 0.05-0.5 μg/kg, 0.05-1 μg/kg, 0.1-0.5 μg/kg, 0.1-1 μg/kg, 0.5-5 μg/kg, 1-10 μg/kg, 5-50 μg/kg, or 10-100 μg/kg. In certain embodiments, the amount of IL-12 intratumorally administered to a cancer patient, can range from 0.01, 0.05, 0.1, 0.5, or 1 μg/kg to 1.5, 5, 10, 25, 50, or 100 μg/kg. In some embodiments, the amount of IL-12 intratumorally administered to a cancer patient, can range from 0.01, 0.05, or 0.1 μg/kg to 0.5, 1, 1.5, or 2 μg/kg.

In another embodiment, for IL-2, IL-7, IL-10, or IL-15, the amount of the cytokine intratumorally administered to a cancer patient can be in the range of 0.1 μg/kg to 1 mg/kg, such as in the range of 0.1-1 μg/kg, 0.1-10 μg/kg, 0.5-5 μg/kg, 0.5-50 μg/kg, 1-10 μg/kg, 1-50 μg/kg, 1-100 μg/kg, 5-50 μg/kg, 5-100 μg/kg, 10-100 gg/kg, 50-500 μg/kg, or 100 μg/kg to 1 mg/kg. In certain embodiments, the amount of IL-12 intratumorally administered to a cancer patient, can range from 0.1, 0.5, 1, 1.5 μg/kg to 5, 10, 25, 50, 100, 500, or 1,000 μg/kg.

In methods described herein involving combination therapy and administration (e.g., conjoint administration) of a STING agonist and a cytokine with an immune checkpoint inhibitor, wherein the immune checkpoint inhibitor is administered systematically, the immune checkpoint inhibitor can be administered to a cancer patient in amounts that have been approved by a relevant regulatory authority, such as the U.S. Food and Drug Administration. For example, in some embodiments, the immune checkpoint inhibitor is a PD-1 inhibitor, such as pembrolizumab (Keytruda®) or nivolumab (Opdivo®) and is administered intravenously at a dose of 100-400 mg, such as 200 mg for pembrolizumab or 240 mg for nivolumab. In another example, the immune checkpoint inhibitor is a PD-L1 inhibitor, such as atezolizumab (Tecentriq®), avelumab (Bavencio®), or durvalumab (Imfinzi®), and is administered intravenously at a dose of 400-2,000 mg, such as 840-1680 mg for atezolizumab, 800 mg for avelumab, or 1500 mg or 10 mg/kg for durvalumab. In another example, the immune checkpoint inhibitor is a CTLA-4 inhibitor, such as ipilimumab (Yervoy®), and is administered intravenously at a dose of 2-5 mg/kg, such as 3 mg/kg.

### 6.7. STING Agonist Dosing Regimens with Improved Safety Profiles

In some embodiments, the STING agonist is administered under a dosing schedule that includes a priming dose followed by multiple maintenance doses. A priming dose refers to a dose that is administered at lower doses than the maintenance doses to increase the tolerance of the body for a particular active agent (e.g., a STING agonist). It has been found that administration of a priming dose of the STING agonist improves the safety profile of the STING agonist and allows the compound to be delivered at higher maintenance dosage levels than would otherwise be tolerated. In general, the priming dosage amount will be less than the maintenance doses over the course of a given dosing cycle.

Accordingly, the disclosure provides novel dosing schedules for STING agonists based on specific dosing schedules requiring administration of a priming dose followed by administration of maintenance doses. In particular embodiments, the novel STING agonist dosing schedules described herein also involve conjoint administration with a cytokine, and optionally, one or more immune checkpoint inhibitors, particularly PD-1 inhibitor, a PD-L1 inhibitor, or a CTLA-4 inhibitor, as disclosed herein. Using the combination of the STING agonist priming/maintenance dosing regimen conjointly with a cytokine is expected to provide an improved therapeutic index.

Particular STING agonists that can be administered using the disclosed priming/maintenance dosing schedules are described above. In some embodiments, the STING agonist to be administered with the disclosed priming/maintenance dosing schedule is Compound A. In some embodiments, the STING agonist to be administered with the disclosed priming/maintenance dosing schedule is not Compound A. In some embodiments, the STING agonist to be administered with the disclosed priming/maintenance dosing schedule is Compound B. In some embodiments, the STING agonist to be administered with the disclosed priming/maintenance dosing schedule is Compound C. In some embodiments, the STING agonist to be administered with the disclosed priming/maintenance dosing schedule is Compound D. In some embodiments, the STING agonist to be administered with the disclosed priming/maintenance dosing schedule is Compound E. In some embodiments, the STING agonist to be administered with the disclosed priming/maintenance dosing schedule is Compound F. In some embodiments, the STING agonist to be administered with the disclosed priming/maintenance dosing schedule is Compound G. In certain embodiments, the STING agonist to be administered with the disclosed priming/maintenance dosing schedule is administered as part of an ADC, such as those described herein.

In some embodiments, the priming dose of the STING agonist can be administered in a quantity (by weight) that is 2- to 100-fold less than the individual maintenance doses in a given dosing cycle. For instance, the priming dose can be administered in a quantity that is 2- to 70-fold less than, 2- to 50-fold less than, 2- to 30-fold less than, 2- to 20-fold less than, 2- to 10-fold less than, 10 to 50-fold less than, 10- to 30-fold less than, 10- to 20-fold less, or 20- to 30-fold less than the maintenance doses in a given cycle. In some embodiments, the priming dose can be administered in a quantity that is 2- to 4-fold less than the maintenance doses in a given cycle. In some embodiments, the priming dose can be administered in a quantity that is 2- to 5-fold less than the maintenance doses in a given cycle. In some embodiments, the priming dose can be administered in a quantity that is 2- to 8-fold less than the maintenance doses in a given cycle. In some embodiments, the priming dose can be administered in a quantity that is 3- to 5-fold less than the maintenance doses in a given cycle. In some embodiments, the priming dose can be administered in a quantity that is 3- to 8-fold less than the maintenance doses in a given cycle. In some embodiments, the priming dose can be administered in a quantity that is 4- to 8-fold less than the maintenance doses in a given cycle.

In some embodiments, the priming dose can be delivered at a dose that is about 2-fold less than the maintenance doses over the course of a dosing cycle. In some embodiments, the priming dose can be delivered at a dose that is about 3-fold less than the maintenance doses over the course of a dosing cycle. In some embodiments, the priming dose can be delivered at a dose that is about 4-fold less than the maintenance doses over the course of a dosing cycle. In some embodiments, the priming dose can be delivered at a dose that is about 5-fold less than the maintenance doses over the course of a dosing cycle. In some embodiments, the priming dose can be delivered at a dose that is about 10-fold less than the maintenance doses over the course of a dosing cycle. In some embodiments, the priming dose can be delivered at a dose that is about 15-fold less than the maintenance doses over the course of a dosing cycle. In some embodiments, the priming dose can be delivered at a dose that is about 20-fold less than the maintenance doses over the course of a dosing cycle. In some embodiments, the priming dose can be delivered at a dose that is about 50-fold less than the maintenance doses over the course of a dosing cycle. In some embodiments, the priming dose can be delivered at a dose that is about 100-fold less than the maintenance doses over the course of a dosing cycle.

It should be understood that the above relative amounts of priming dose to the individual maintenance doses can be expressed as a ratio. For instance, in an embodiment where the priming dose is administered at a dose that is about 2-fold less than the maintenance doses, a dosing regimen that involves a 1:2 ratio of priming dose to individual maintenance doses is described. Accordingly, in certain embodiments, the present disclosure provides a method of treating cancer comprising administering the combination of a STING agonist and a cytokine to a patient in need thereof, wherein the STING agonist is administered according to a dosing regimen that includes a 1:2 to 1:100 ratio of priming dose to individual maintenance doses, such as a ratio of 1:2, 2:5, 3:8, 1:3, 2:7, 1:4, 1:5, 1:6, 1:8, 1:9, 1:10, 1:11, 1:12, 1:15, 1:20, 1:30, 1:50, 1:75, or 1:100, including ranges created by these ratios, such as 1:2 to 1:3, 1:2 to 1:4, 1:2 to 1:5, 1:2 to 1:8, 1:2 to 1:10, 1:4 to 1:8, 1:4 to 1:10, 1:4 to 1:15, 1:4 to 1:20, 1:8 to 1:10, 1:8 to 1:15, 1:8 to 1:20, 1:8 to 1:30, 1:10 to 1:15, 1:10 to 1:20, 1:10 to 1:30, 1:10 to 1:50, 1:20 to 1:30, 1:20 to 1:50, 1:20 to 1:75, 1:20 to 1:100, 1:30: to 1:50, 1:30 to 1:75, 1:30 to 1:100, 1:50 to 1:75, 1:50 to 1:100, or 1:75 to 1:100.

In some embodiments, the present disclosure provides a method of treating cancer comprising administering the combination of a STING agonist and a cytokine to a patient in need thereof, wherein the STING agonist is administered according to a dosing regimen that includes a 1:4 or 1:5 ratio of priming dose to individual maintenance doses, or a ratio in the range of 1:3 to 1:6, such as 1:3 to 1:5, 1:4 to 1:6, or 1:4 to 1:5. In other embodiments, the ratio is 1:8 or 1:10, or a ratio in the range of 1:5 to 1:15, such as 1:6 to 1:12, 1:8 to 1:12, 1:8 to 1:10, or 1:9 to 1:10.

In some embodiments, the priming dose can be administered on day 1 of a treatment cycle and the maintenance doses can be administered thereafter at a dosing schedule as described above. The first maintenance dose can be administered at least 2 days following the administration of the priming dose, i.e., on day 3. For instance, the first maintenance dose can be administered 2, 3, 4, 5, 6, 7, 8, 9, or 10 days following administration of the priming dose.

In one embodiment, the dosing cycle comprises administering a priming dose of the STING agonist on day 1 of a treatment cycle followed by administering maintenance doses of the STING agonist on days 8, 15 and 22 (i.e., the first day of weeks 2, 3 and 4) of the treatment cycle, followed by a period of one week (i.e., week 5) where the STING agonist is not administered to the patient. The maintenance dosing cycle can be repeated, or a modified maintenance dosing schedule can be employed.

In another embodiment, the dosing cycle comprises administering a priming dose of the priming dose on day 1 of a treatment cycle followed by administering maintenance doses of the STING agonist on days 8 and 22 of the dosing schedule (i.e., biweekly dosing). The maintenance dosing cycle can be repeated or a modified maintenance dosing schedule can be employed.

### 7. EXAMPLES

### Example 1. Combination Studies Involving a STING Agonist, an Immune Checkpoint Inhibitor, and a Cytokine

The anti-tumor effect of a triple combination of a STING agonist (Compound A), an immune checkpoint inhibitor (anti-PD-L1 antibody), and cytokines IL-2, IL-12, or IL-15 was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 7 and mice were regrouped so that each group had similar average tumor volumes. Each group contained 5 mice. Subsequently and on the same day 7 and on days 11, and 15, mice were mock treated with PBS or treated intraperitoneally with 200 μg of an anti-PD-L1 antibody and intratumorally on their right site (primary) with 1 pg of Compound A alone or in combination with IL-2 (5 μg), IL-12 (2 μg), or IL-15 (5 μg). Cytokines and Compound A were administered in a vehicle of 50 μL of PBS containing 0.2 mg/mL bovine serum albumin. Tumor volumes were measured every 2-3 days and survival was monitored daily. Anti-PD-L1 antibody (BE0101) was purchased from BioXcell (Lebanon, N.H.), IL-2 (212-12), IL-12 (210-12), and IL-15 (210-15) were purchased from PeproTech (Rocky Hill, N.J.).

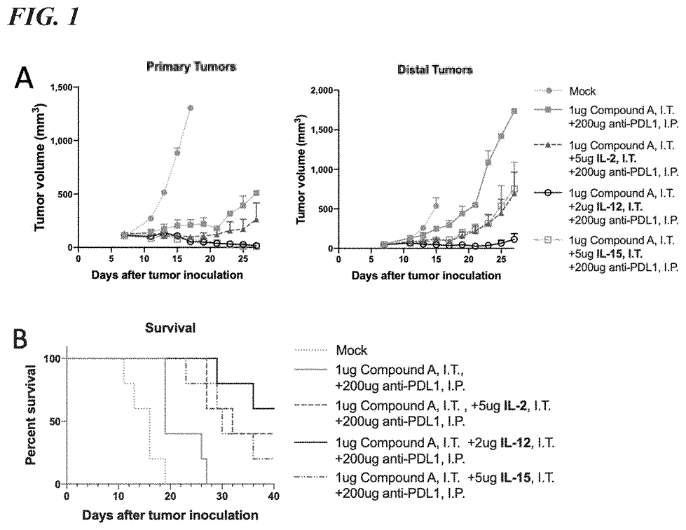

An increase in anti-tumor effect on the primary tumor was observed with a triple combination of Compound A, anti-PD-L1 antibody, and cytokines IL-2, IL-12, or IL-15 when compared to treatment with the double combination of Compound A and anti-PD-L1 antibody (panel A of FIG. 1).

A more dramatic increase in anti-tumor effect on the distal untreated tumor (i.e., an abscopal effect) was observed when the triple combination of Compound A, anti-PD-L1 antibody, and cytokines IL-2, IL-12, or IL-15 was used when compared to treatment with the double combination of Compound A and anti-PD-L1 antibody (panel A of FIG. 1). This dramatically increased anti-tumor effect was also reflected in the increased mouse survival for the triple combinations (panel B of FIG. 1).

### Example 2. More Combination Studies Involving a STING Agonist, an Immune Checkpoint Inhibitor, and a Cytokine

The anti-tumor effect of a triple combination of a STING agonist (Compound A), an immune checkpoint inhibitor (anti-PD-L1 antibody), and cytokines IL-7 or IL-10 was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 7 and mice were regrouped so that each group had similar average tumor volumes. Each group contained 5 mice. Subsequently and on the same day 7 and on days 11, and 15, mice were mock treated with PBS or treated intraperitoneally with 200 μg of an anti-PD-L1 antibody and intratumorally on their right site (primary) with 1 μg of Compound A alone or in combination with IL-7 (5 μg) or IL-10 (5 μg). Cytokines and Compound A were administered in a vehicle of 50 μL of PBS containing 0.2 mg/mL bovine serum albumin. Tumor volumes were measured every 2-3 days and survival was monitored daily. Anti-PD-L1 antibody (BE0101) was purchased from BioXcell (Lebanon, N.H.), IL-7 (217-17) and IL-10 (210-10) were from PeproTech (Rocky Hill, N.J.).

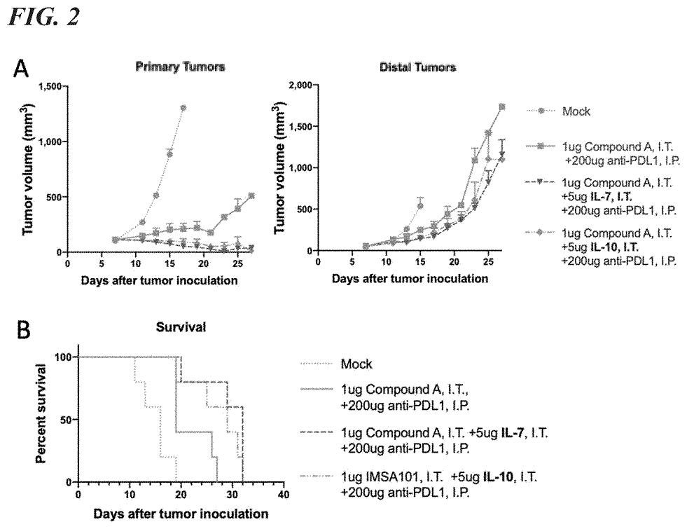

A significant increase in anti-tumor effect on the primary tumor was observed with a triple combination of Compound A, anti-PD-L1 antibody, and cytokines IL-7 or IL-10 when compared to treatment with the double combination of Compound A and anti-PD-L1 antibody (panel A of FIG. 2).

A modest increase in anti-tumor effect on the distal untreated tumor (i.e., an abscopal effect) was also observed when the triple combination of Compound A, anti-PD-L1 antibody, and cytokines IL-7 or IL-10 was used when compared to treatment with the double combination of Compound A and anti-PD-L1 antibody (panel A of FIG. 2). This increased anti-tumor effect was also reflected in the increased mouse survival for the triple combinations (panel B of FIG. 2).

### Example 3. Further Combination Studies Involving a STING Agonist, an Immune Checkpoint Inhibitor, and IL-12

The anti-tumor effect of a triple combination of a STING agonist (Compound A), an immune checkpoint inhibitor (anti-PD-L1 antibody), and various doses of IL-12 (50 ng, 200 ng, and 1 μg) was examined in comparison to the double combination of Compound A and anti-PD-L1 antibody or IL-12 and anti-PD-L1 antibody.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 6 and mice were regrouped so that each group had similar average tumor volumes. Each group contained 5 mice. Subsequently and on the same day 6 and on days 9, 12, and 15, mice were mock treated with PBS or treated intraperitoneally with 200 μg of an anti-PD-L1 antibody and intratumorally on their right site (primary) with 1 μg of Compound A alone; with 50 ng, 200 ng, or 1 μg of IL-12 alone; or with a combination of 1 μg of Compound A and 50 ng, 200 ng, or 1 μg of IL-12. Cytokines and Compound A were administered in a vehicle of 50 μL of PBS containing 0.2 mg/mL bovine serum albumin. Tumor volumes were measured every 2-3 days and survival was monitored daily. Body weight change (%) was measured over time from day 6 before first treatment. Anti-PD-L1 antibody (BE0101) was purchased from BioXcell (Lebanon, N.H.), IL-12 (210-12) was purchased from PeproTech (Rocky Hill, N.J.).

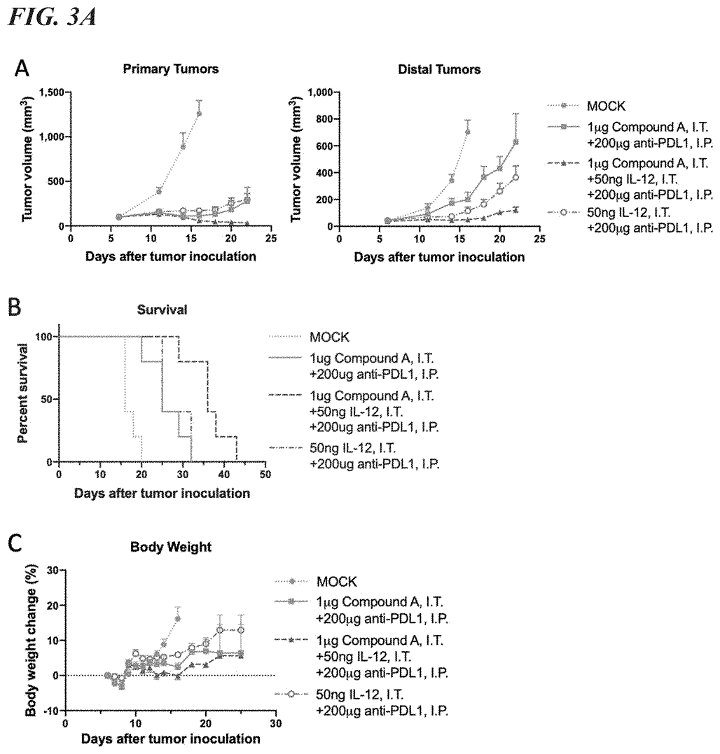

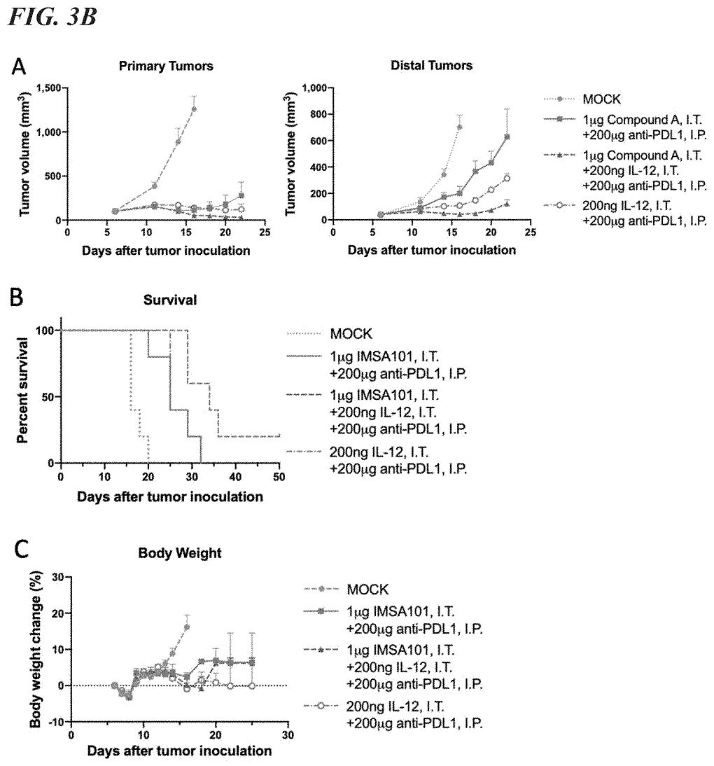

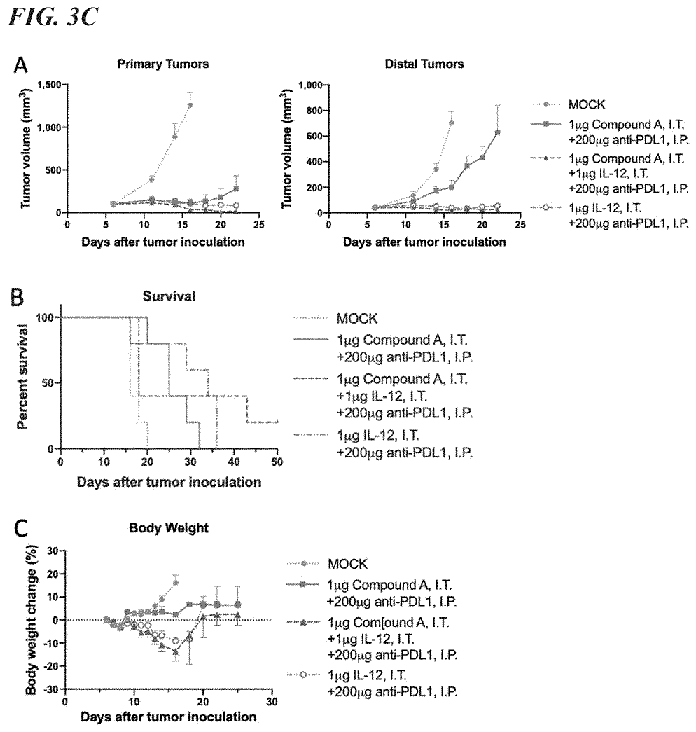

An increase in anti-tumor effect on the primary tumor was observed with a triple combination of Compound A, anti-PD-L1 antibody, and IL-12 when compared to treatment with the double combination of Compound A and anti-PD-L1 antibody or IL-12 and anti-PD-L1 antibody (panel A of each of FIGS. 3A, 3B, and 3C).

A significant increase in anti-tumor effect on the distal untreated tumor (i.e., an abscopal effect) was observed when the triple combination of Compound A, anti-PD-L1 antibody, and IL-12 (50 ng or 200 ng) was used when compared to treatment with the double combination of Compound A and anti-PD-L1 antibody or IL-12 and anti-PD-L1 antibody (panel A of each of FIGS. 3A and 3B). These increased anti-tumor effects were also reflected in the increased mouse survival for the triple combinations (panel B of each of FIGS. 3A and 3B). A significant abscopal effect was also observed for the triple combination of Compound A, anti-PD-L1 antibody, and IL-12 (1 μg) when compared to treatment with the double combination of Compound A and anti-PD-L1 antibody (panel A of FIG. 3C). However, no further abscopal effect was observed in this triple combination when compared to the double combination of IL-12 (1 μg) and anti-PD-L1 antibody, likely due to the saturated effect of IL-12 at this higher dose. The triple combination of Compound A, anti-PD-L1 antibody, and IL-12 (1 μg) resulted in increased survival for certain of the mice but also resulted in early deaths for other mice, possibly due to toxicity associated with the higher dose of IL-12 in the triple combination (panel B of FIG. 3C).

All treatment groups, except those receiving combinations using 1 μg of IL-12, exhibited initial transient and minimal (<5%) body weight reduction followed by rapid recovery, illustrating the low toxicity of the various combinations (panel C of each of FIGS. 3A and 3B). But combination treatments employing 1 μg of IL-12 caused significant weight reduction (up to 15%), indicating increased mouse toxicity for this dose (panel C of FIG. 3C).

### Example 4. Titration Studies Involving a STING Agonist, an Immune Checkpoint Inhibitor, and IL-12

The anti-tumor effect of a triple combination of a STING agonist (Compound A), an immune checkpoint inhibitor (anti-PD-L1 antibody), and various doses of IL-12 (3 ng, 10 ng, and 30 ng) was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 9 and mice were regrouped so that each group had similar average tumor volumes. Each group contained 5 mice. Subsequently and on the same day 9 and on days 12, 15, and 18, mice were mock treated with PBS or treated intraperitoneally with 200 μg of an anti-PD-L1 antibody and intratumorally on their right site (primary) with 1 μg of Compound A alone or in combination with 3 ng, 10 ng, or 30 ng of IL-12. Cytokines and Compound A were administered in a vehicle of 50 μL of PBS containing 0.2 mg/mL bovine serum albumin. Tumor volumes were measured every 2-3 days and survival was monitored daily. Body weight change (%) was measured over time from day 9 before first treatment. Anti-PD-L1 antibody (BE0101) was purchased from BioXcell (Lebanon, N.H.), IL-12 (210-12) was purchased from PeproTech (Rocky Hill, N.J.).

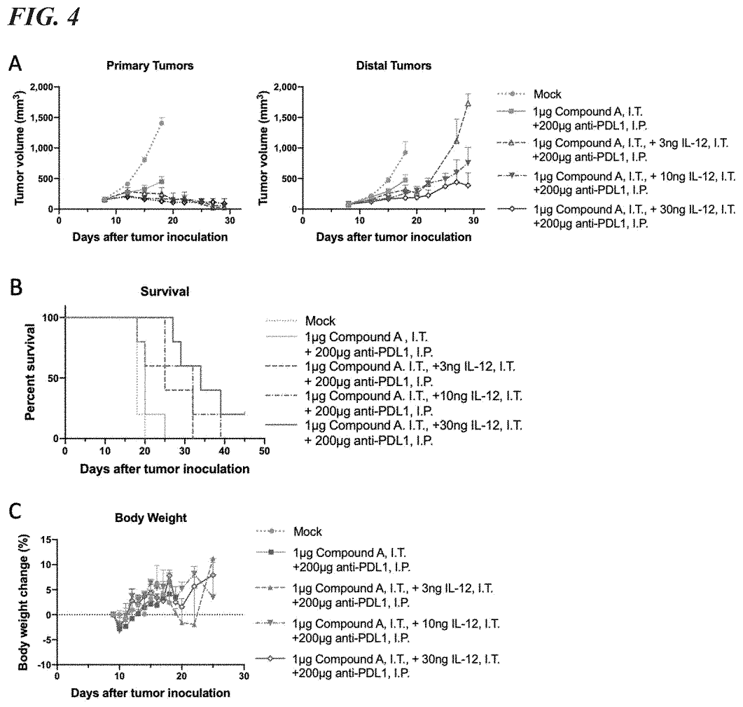

Compared to the double combination of Compound A and anti-PD-L1 antibody, increased anti-tumor effect on the primary tumor was observed when a triple combination of Compound A, anti-PD-L1 antibody, and 3 ng, 10 ng, or 30 ng of IL-12 was used (panel A of FIG. 4).

A dose-responsive anti-tumor effect on the distal untreated tumor (i.e., an abscopal effect) was observed when the triple combination of Compound A, anti-PD-L1 antibody, 3 ng, 10 ng, or 30 ng of IL-12 was used (panel A of FIG. 4). This dose-responsive anti-tumor effect was also reflected in the increased mouse survival for the various triple combinations (panel B of FIG. 4).

The initial transient and minimal (<5%) body weight reduction followed by rapid recovery illustrated the low toxicity of the various combinations (panel C of FIG. 4).

### Example 5. Combination Studies Involving a STING Agonist, an Immune Checkpoint Inhibitor, and IL-12-Fc

The anti-tumor effect of combinations of a STING agonist (Compound A), an immune checkpoint inhibitor (anti-PD-L1 antibody), and IL-12-Fc was examined.

Female C57BL6 mice (Jackson Laboratory) at the age of 7-8 weeks were implanted subcutaneously with 10′ B16F10 (ATCC CRL-6475) melanoma cells into the right flank (primary tumor) on day 0 and left flank (distal tumor) on day 2. Tumors were measured on day 9 and mice were regrouped so that each group had similar average tumor volumes. Each group contained 5 mice. Subsequently and on the same day 9 and on days 12, and 15, mice were mock treated or treated intratumorally on their right flank (primary) with 1 μg of Compound A and 50 ng of IL-12-Fc, or intraperitoneally with 200 μg of an anti-PD-L1 antibody and intratumorally on their right site (primary) with 1 μg of Compound A alone, 50 ng of IL-12-Fc alone, or a combination of 1 μg of Compound A and 50 ng of IL-12-Fc. Cytokines and Compound A were administered in a vehicle of 50 μL of PBS containing 0.2 mg/mL bovine serum albumin. Tumor volumes were measured every 2-3 days and survival was monitored daily. Body weight change (%) was measured over time from day 9 before first treatment. Anti-PD-L1 antibody (BE0101) was purchased from BioXcell (Lebanon, N.H.).

The IL-12-Fc protein comprised two subunits of mouse interleukine-12 (p35 and p40), each fused to the Fc domain of human IgG1, with the following amino acid sequences:

(SEQ ID NO: 1)
Mu IL-12 p35-Lmker-huIgG1 Fc (Hole):
MCQRYLLFLATLALLNHLSLARVIPVSGPARCLSQSRNLLKTTDDMVKTA
REKLKHYSCTAEDIDHEDITRDQTSTLKTCLPLELHKNESCLATRETSST
TRGSCLPPQKTSLMMTLCLGSIYEDLKMYQTEFQAINAALQNHNHQQIIL
DKGMLVAIDELMQSLNHNGETLRQKPPVGEADPYRVKMKLCILLHAFSTR
VVTINRVMGYLSSA [symbol] [symbol]
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVCTLPPSREEMTKNQVSLSCAVKGFYPSDIAVEW
ESNGQPENNYKTTPPVLDSDGSFFLVSKLTVDKSRWQQGNVFSCSVMHEA
LHNHYTQKSLSLSPGK
(SEQ ID NO: 2)
Mu IL-12 p40-Lmker-huIgG1 Fc (Knob):
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTHLKCEAPNYSGRITCSWLVQRNMDIK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRS [symbol]
PELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQ
VSLWCLVKGFYPSDIAVEWESNGQPENNYKTTPPVLDSDGSFFLYSKLTV
DKSRWQQGNVFSCSVMHEALHNHYTQKSLSLSPGK

Signal peptides are underlined, and linker sequences are bold+italic.

cDNA encoding each subunit was cloned into pcDNA3.4-TOPO vector (Invitrogen, A14697). To express IL-12-Fc, both plasmids were transfected into CHO (ATCC (CCL-61) cells using ExpiFectamine™ CHO Transfection Kit (Gibco, A29129) following the manufacturer's protocol. Seven days after transfection, the culture media were collected and the IL-12-Fc protein was loaded onto a 5 ml HiTrap protein A column (GE, 17-0403-01) on a Bio-Rad NGC Chromatography system (Bio-Rad, NGC Quest 10, 7880001). The column was washed with 50 ml of PBS and eluted with 25 ml of 0.1M Glycine, pH2.5. The eluate was concentrated and further purified on a gel filtration column (Bio-Rad, ENrich 650, 7801650) equilibrated with PBS.

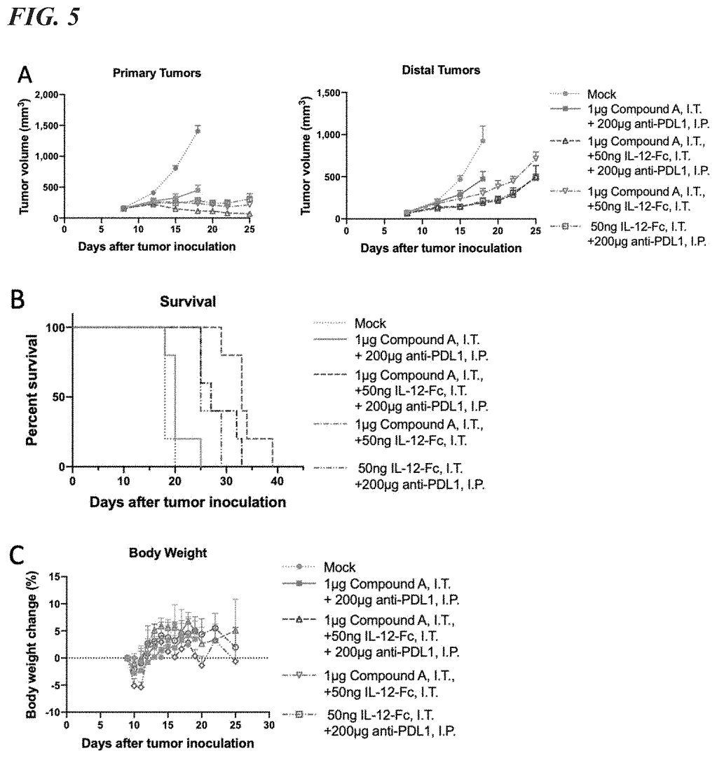

Improved anti-tumor effect was observed in primary tumors for a triple combination of Compound A, anti-PD-L1 antibody, and IL-12-Fc in comparison to the double combination of Compound A and IL-12-Fc, anti-PD-L1 antibody and IL-12-Fc, or Compound A and anti-PD-L1 antibody. An increase in anti-tumor effect on the distal untreated tumor (i.e., an abscopal effect) was also improved in the triple combination as well as in the double combination of anti-PDL1 antibody and IL-12-Fc (panel A of FIG. 5). However, an improved anti-tumor effect for the triple combination is reflected in the increased mouse survival (panel B of FIG. 5).

The initial transient and minimal (<5%) body weight reduction followed by rapid recovery illustrated the low toxicity of the various combinations (panel C of FIG. 5).

### Example 6. Titration Studies Involving a STING Agonist, an Immune Checkpoint Inhibitor, and IL-12-Fc

The anti-tumor effect of a triple combination of a STING agonist (Compound A), an immune checkpoint inhibitor (anti-PD-L1 antibody), and IL-12-Fc at various doses was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 9 and mice were regrouped so that each group had similar average tumor volumes. Each group contained 5 mice. Subsequently and on the same day 9 and on days 12, 15, and 18, mice were mock treated with PBS or treated intraperitoneally with 200 μg of an anti-PD-L1 antibody and intratumorally on their right site (primary) with 1 μg of Compound A alone or in combination with 5 ng, 17 ng, or 50 ng of IL-12-Fc. Cytokines and Compound A were administered in a vehicle of 50 μL of PBS containing 0.2 mg/mL bovine serum albumin. Tumor volumes were measured every 2-3 days and survival was monitored daily. Anti-PD-L1 antibody (BE0101) was purchased from BioXcell (Lebanon, N.H.). The IL-12-Fc protein was obtained as described above.

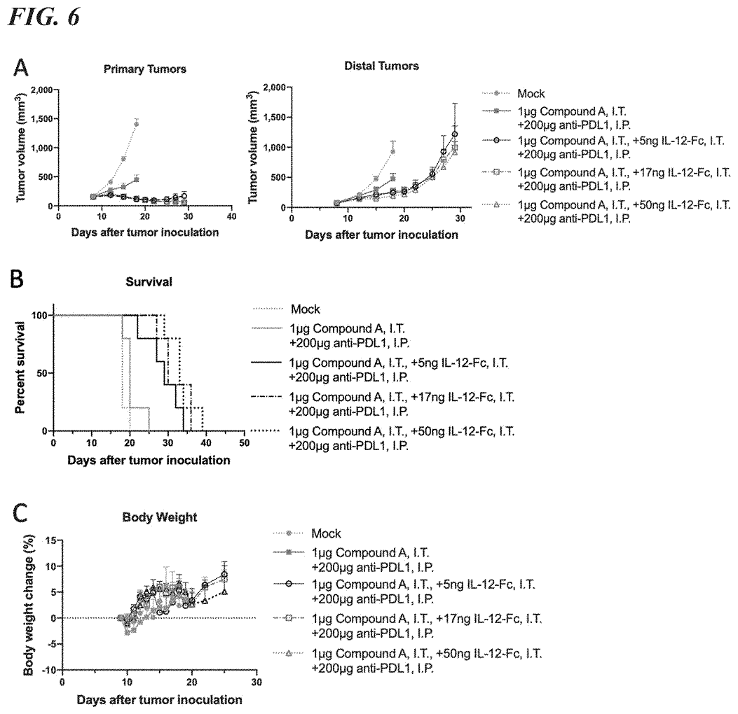

Comparable increased anti-tumor effects on the primary tumor and distal untreated tumor were observed for the various triple combinations of Compound A, anti-PD-L1 antibody, and IL-12-Fc in comparison to the double combination of Compound A and anti-PD-L1 antibody (panel A of FIG. 6). Also, an improved anti-tumor effect for the triple combinations was reflected in the increased mouse survival in comparison to the double combination of Compound A and anti-PD-L1 antibody (panel B of FIG. 6).

The initial transient and minimal (<5%) body weight reduction followed by rapid recovery illustrated the low toxicity of the various combinations (panel C of FIG. 6).

### Example 7. Combination Studies Involving a STING Agonist, an Immune Checkpoint Inhibitor, and Interleukins IL-12-Fc or mIL-12-MSA-Lumican

The anti-tumor effect of combinations of a STING agonist (Compound A), an immune checkpoint inhibitor (anti-PD-L1 antibody), and IL-12-Fc or mIL-12-MSA-Lumican was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 9 and mice were regrouped so that each group had similar average tumor volumes. Each group contained 5 mice. Subsequently and on the same day 9 and on days 12, and 15, mice were mock treated with PBS or treated intraperitoneally with 200 μg of an anti-PD-L1 antibody and intratumorally on their right flank (primary) with 1 μg of Compound A alone or in combination with IL-12-Fc (30 ng) or mIL-12-MSA-Lumican (20 ng, 60 ng, or 200 ng). Cytokines and Compound A were administered in a vehicle of 50 μL of PBS containing 0.5% mouse serum. Tumor volumes were measured every 2-3 days and survival was monitored daily. Anti-PD-L1 antibody (BE0101) was purchased from BioXcell (Lebanon, N.H.).

The fusion protein designated mIL-12-MSA-Lumican contains (from N-terminal to C-terminal) murine interleuline-12, murine serum albumin, and Lumican, a collagen-binding moiety which anchors the molecule in tumors. To express this protein, the encoding cDNA was cloned into pcDNA3.4-TOPO vector (Invitrogen, A14697) and transfected into CHO cells using ExpiFectamine™ CHO Transfection Kit (Gibco, A29129) following the manufacturer's protocol. Seven days after transfection, the culture media were collected and loaded onto a 5 ml HisTrap Excel column (Cytiva, 17371206) on a Bio-Rad NGC Chromatography system (Bio-Rad, NGC Quest 10, 7880001). The column was washed with 50 ml of PBS and eluted with 25 ml of 0.5M Imidazole in 50 mM Tris-HCl solution (pH 8.0). The eluent was concentrated and further purified on a gel filtration column (Bio-Rad, ENrich 650, 7801650) equilibrated with PBS.

(SEQ ID NO: 3)
Amino acid sequence of mIL12-MSA-Lumican:
MWELEKDVYVVEVDWTPDAPGETVNLTCDTPEEDDITWTSDQRHGVIGSG
KTLTITVKEFLDAGQYTCHKGGETLSHSHLLLHKKENGIWSTEILKNFKN
KTFLKCEAPNYSGRFTCSWLVQRNMDLKFNIKSSSSSPDSRAVTCGMASL
SAEKVTLDQRDYEKYSVSCQEDVTCPTAEETLPIELALEARQQNKYENYS
TSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSS
CSKWACVPCRVRSGGSGGGSGGGSGGGSRVIPVSGPARCLSQSRNLLKTT
DDMVKTAREKLKHYSCTAEDIDHEDITRDQTSTLKTCLPLELHKNESCLA
TRETSSTTRGSCLPPQKTSLMMTLCLGSIYEDLKMYQTEFQAINAALQNH
NHQQIILDKGMLVAIDELMQSLNHNGETLRQKPPVGEADPYRVKMKLCIL
LHAFSTRVVTINRVMGYLSSAGSGGGSEAHKSEIAHRYNDLGEQHFKGLV
LIAFSQYLQKCSYDEHAKLVQEVTDFAKTCVADESAANCDKSLHTLFGDK
LCAIPNLRENYGELADCCTKQEPERNECFLQHKDDNPSLPPFERPEAEAM
CTSFKENPTTFMGHYLHEVARRHPYFYAPELLYYAEQYNEILTQCCAEAD
KESCLTPKLDGVKEKALVSSVRQRMKCSSMQKFGERAFKAWAVARLSQTF
PNADFAEITKLATDLTKVNKECCHGDLLECADDRAELAKYMCENQATISS
KLQTCCDKPLLKKAHCLSEVEHDTMPADLPAIAADFVEDQEVCKNYAEAK
DVFLGTFLYEYSRRHPDYSVSLLLRLAKKYEATLEKCCAEANPPACYGTV
LAEFQPLVEEPKNLVKTNCDLYEKLGEYGFQNAILVRYTQKAPQVSTPTL
VEAARNLGRVGTKCCTLPEDQRLPCVEDYLSAILNRVCLLHEKTPVSEHV
TKCCSGSLVERRPCFSALTVDETYVPKEFKAETFTFHSDICTLPEKEKQI
KKQTALAELVKHKPKATAEQLKTVMDDFAQFLDTCCKAADKDTCFSTEGP
NLVTRCKDALAGGGSGGGSQYYDYDIPLFMYGQISPNCAPECNCPHSYPT
AMYCDDLKLKSVPMVPPGIKYLYLRNNQIDHIDEKAFENVTDLQWLILDH
NLLENSKIKGKVFSKLKQLKKLHINYNNLTESVGPLPKSLQDLQLTNNKI
SKLGSFDGLVNLTFIYLQHNQLKEDAVSASLKGLKSLEYLDLSFNQMSKL
PAGLPTSLLTLYLDNNKISNIPDEYFKRFTGLQYLRLSHNELADSGVPGN
SFNISSLLELDLSYNKLKSIPTVNENLENYYLEVNELEKFDVKSFCKILG
PLSYSKIKHLRLDGNPLTQSSLPPDMYECLRVANEITVNGGGSHHHHHH

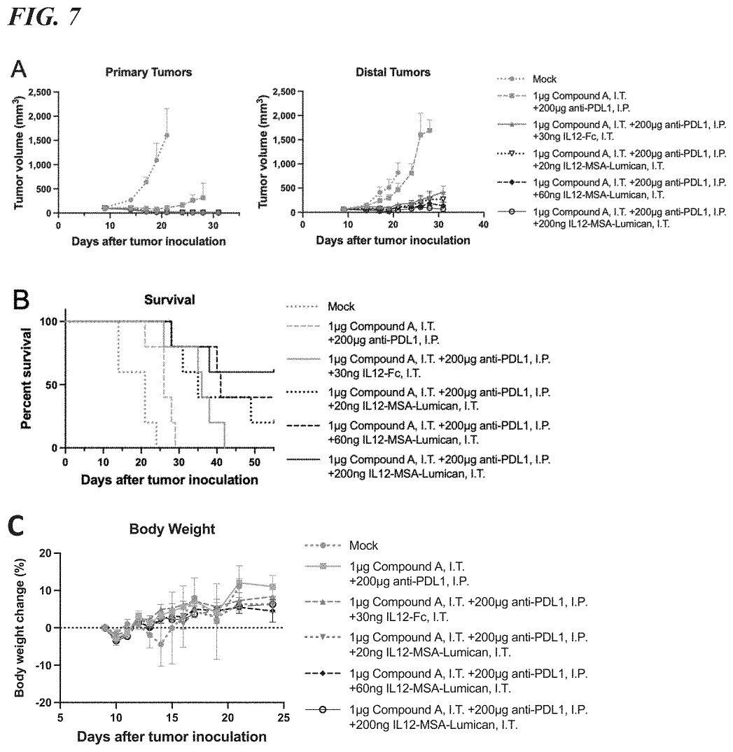

Double combination of Compound A and anti-PD-L1 antibody showed anti-tumor effect in primary tumors but limited anti-tumor effect in distal tumors. In contrast, triple combination of Compound A, anti-PD-L1 antibody, and IL-12-Fc or mIL-12-MSA-Lumican showed anti-tumor effect in primary and distal tumors (panel A of FIG. 7). Mice survival showed to be dose-dependent in groups treated with mIL-12-MSA-Lumican (panel B of FIG. 7).

All triple combination treatment groups exhibited comparable body weight variation and similar to the pattern observed in the mock treatment group or the dual combination of Compound A and anti-PD-L1 antibody, illustrating the low toxicity of the various combinations (panel C of FIG. 7).

### Example 8. Further Combination Studies Involving a STING Agonist, an Immune Checkpoint Inhibitor, and Interleukin mIL-12-MSA-Lumican

The anti-tumor effect of various combinations of a STING agonist (Compound A), an immune checkpoint inhibitor (anti-PD-L1 antibody), and interleukin mIL-12-MSA-Lumican was examined.

Female C57BL6 mice (Jackson Laboratory) at the age of 7-8 weeks were implanted subcutaneously with 10⁶ B16F10 (ATCC CRL-6475) melanoma cells into the right flank (primary tumor) on day 0 and left flank (distal tumor) on day 2. Tumors were measured on day 9 and mice were regrouped so that each group had similar average tumor volumes. Each group contained 5 mice. Subsequently and on the same day 9 and on days 12, and 15, mice were mock treated with PBS or: 1) treated intratumorally on their right flank (primary) with 60 ng of mIL-12-MSA-Lumican and treated intraperitoneally with 200 μg of an anti-PD-L1 antibody; 2) treated intratumorally on their right flank (primary) with 1 μg of Compound A and treated intraperitoneally with 200 μg of an anti-PD-L1 antibody; 3) treated intratumorally on their right flank (primary) with 1 μg of Compound A and treated intratumorally on their right flank (primary) with 60 ng of mIL-12-MSA-Lumican or 4) treated intratumorally on their right flank (primary) with 1 μg of Compound A and with 60 ng of mIL-12-MSA-Lumican and treated intraperitoneally with 200 μg of an anti-PD-L1 antibody. Cytokines and Compound A were administered in a vehicle of 50 μL of PBS containing 0.5% mouse serum. Tumor volumes were measured every 2-3 days and survival was monitored daily. Anti-PD-L1 antibody (BE0101) was purchased from BioXcell (Lebanon, N.H.).

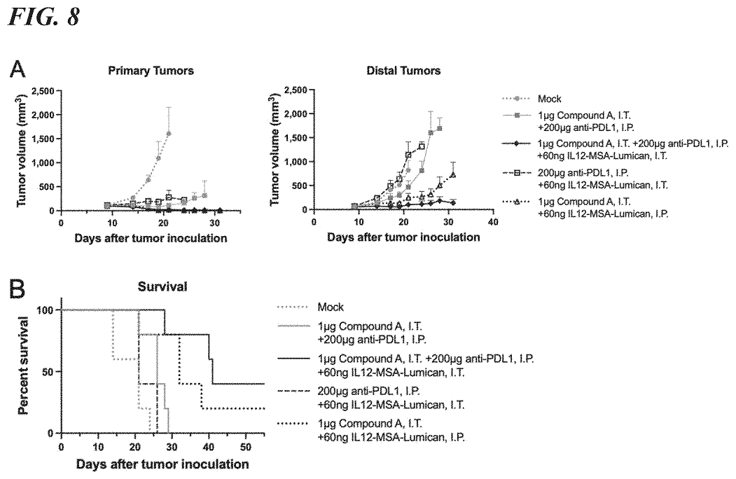

Double combination of mIL-12-MSA-Lumican and anti-PD-L1 antibody showed partial anti-tumor effect in primary tumors but no anti-tumor effect in distal tumors. Double combination of Compound A and anti-PD-L1 antibody showed anti-tumor effect in primary tumors but limited anti-tumor effect in distal tumors. Double combination of Compound A and mIL-12-MSA-Lumican showed anti-tumor effect in primary tumors but reduced anti-tumor effect in distal tumors. Triple combination of Compound A, anti-PD-L1 antibody, and mIL-12-MSA-Lumican showed anti-tumor effect in primary and distal tumors (panel A of FIG. 8). Likewise, mice survival was greatest in mice treated with the triple combination versus any of the three double combinations (panel B of FIG. 8).

### Example 9. Tumor Growth in Naive Mice and Pre-treated Mice

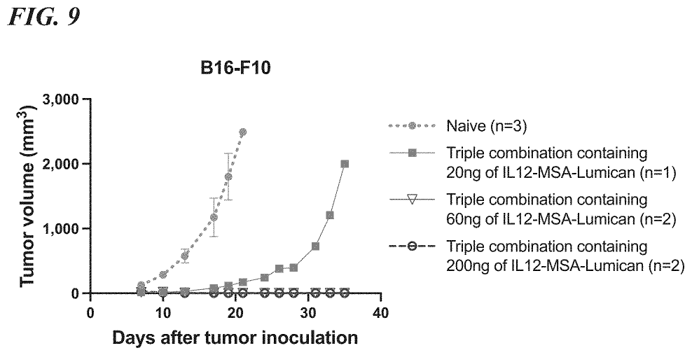

Naïve female C57BL6 mice (Jackson Laboratory) (n=3) or mice that were tumor-free for 35 days following a complete treatment cycle with a triple combination of Compound A, anti-PD-L1 antibody, and mIL-12-MSA-Lumican (20 ng, 60 ng, 200 ng), as described in Example 7, were inoculated subcutaneously with 10⁶ B16F10 (ATCC CRL-6475) melanoma cells and the tumor growth assessed over time. Naïve mice showed tumor progression over time. In contrast, the one mouse treated previously treated with a triple combination of Compound A, anti-PD-L1 antibody, and mIL-12-MSA-Lumican (20 ng) showed slower tumor growth than naïve mice. Mice previously treated with a triple combination of Compound A, anti-PD-L1 antibody, and mIL-12-MSA-Lumican (60 ng) and Compound A, anti-PD-L1 antibody, and mIL-12-MSA-Lumican (200 ng) showed complete tumor suppression (FIG. 9).

1. A method of treating tumors in a cancer patient in need thereof, comprising conjointly administering effective amounts of a STING agonist, a cytokine, and an immune checkpoint inhibitor to the patient, wherein
the STING agonist and the cytokine are intratumorally administered to the patient, and the immune checkpoint inhibitor is systemically administered to the patient;
the STING agonist is a cyclic dinucleotide (CDN);
the cytokine is an interleukin; and
the immune checkpoint inhibitor is an anti-PD-1 antibody, an anti-PD-L1 antibody, or an anti-CTLA-4 antibody.

2. The method of claim 1, wherein the patient exhibits reduced recurrence of the tumors following treatment.

3. A method of augmenting the anti-tumor response of a cancer patient, comprising conjointly administering effective amounts of a STING agonist and a cytokine to the patient, wherein the STING agonist and the cytokine are intratumorally administered to the patient.

4. A method of increasing the population or function of immune cells in a cancer patient, comprising conjointly administering effective amounts of a STING agonist and a cytokine to the patient, wherein the STING agonist or the cytokine is intratumorally administered to the patient.

5. (canceled)

6. The method of claim 1, wherein the method treats tumors distal to the site(s) of intratumoral administration of the STING agonist and the cytokine.

7. A method of reducing recurrence of tumors in a cancer patient in need thereof comprising, conjointly administering effective amounts of a STING agonist, a cytokine, and an immune checkpoint inhibitor to the patient, wherein
the STING agonist and the cytokine are intratumorally administered to the patient, and the immune checkpoint inhibitor is systemically administered to the patient;
the STING agonist is a cyclic dinucleotide (CDN);
the cytokine is an interleukin; and
the immune checkpoint inhibitor is a PD-1 antibody, a PD-L1 antibody, or a CTLA-4 antibody.

8. The method of claim 1, wherein the STING agonist is a 2′3′-CDN.

9. The method of claim 8, wherein the STING agonist is Compound A: 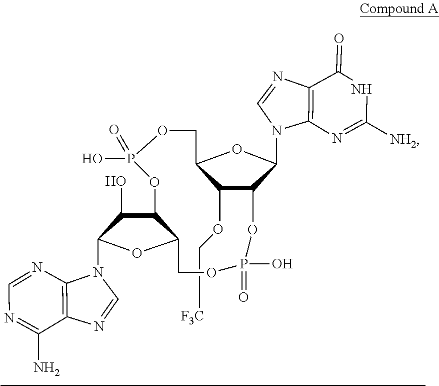
or a pharmaceutically acceptable salt thereof.

10-27. (canceled)

28. The method of claim 1, wherein the immune checkpoint inhibitor is an anti-PD-1 antibody.

29. The method of claim 1, wherein the immune checkpoint inhibitor is an anti-PD-L1 antibody.

30. The method of claim 1, wherein the immune checkpoint inhibitor is an anti-CTLA-4 antibody.

31. (canceled)

32. A method of treating of tumors or preventing the recurrence of tumors in a cancer patient in need thereof, comprising causing a STING agonist, a cytokine, and an immune checkpoint inhibitor to be concurrently present in the patient's body.

33-57. (canceled)

58. A combination therapy suitable for treating tumors in a cancer patient in need thereof, comprising a STING agonist, a cytokine, and an immune checkpoint inhibitor, wherein
the STING agonist and the cytokine are formulated for intratumoral administration to the patient, and the immune checkpoint inhibitor is formulated for systemic administration to the patient;
the STING agonist is a cyclic dinucleotide (CDN);
the cytokine is an interleukin; and
the immune checkpoint inhibitor is an anti-PD-1 antibody, an anti-PD-L1 antibody, or an anti-CTLA-4 antibody.

59-82. (canceled)

83. The method or of claim 1, wherein the interleukin is IL-2, IL-7, IL-10, IL-12, IL-15, or a combination thereof.

84. (canceled)

85. The method of claim 1, wherein the interleukin is IL-2, IL-12, IL-15, or a combination thereof.

86. The method of claim 1, wherein the interleukin is IL-12.

87. (canceled)

88. The method of claim 1, wherein the interleukin is fused to a protein to form a fusion protein.

89-93. (canceled)

94. The method of claim 88, wherein the interleukin is fused to a protein that is not an antibody or antibody fragment.

95. (canceled)

96. The method of claim 94, wherein
the interleukin is fused to a collagen-binding protein;
the collagen-binding protein is lumican; and
the interleukin is IL-12.

97-109. (canceled)

110. A mixture comprising a STING agonist, a cytokine, and an immune checkpoint inhibitor.

111-140. (canceled)


SEQUENCE LISTING (3 sequences)

SEQ ID NO: 1 - protein, 463 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAEPKSCDKTHTCPPCPAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 2 - protein, 567 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 1449 aa, unidentified
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSQYYDYDIPLFMYGQISPNCAPECNCPHSYPTAMYCDDLKLKSVPMVPPGIKYLYLRNNQIDHIDEKAFENVTDLQWLILDHNLLENSKIKGKVFSKLKQLKKLHINYNNLTESVGPLPKSLQDLQLTNNKISKLGSFDGLVNLTFIYLQHNQLKEDAVSASLKGLKSLEYLDLSFNQMSKLPAGLPTSLLTLYLDNNKISNIPDEYFKRFTGLQYLRLSHNELADSGVPGNSFNISSLLELDLSYNKLKSIPTVNENLENYYLEVNELEKFDVKSFCKILGPLSYSKIKHLRLDGNPLTQSSLPPDMYECLRVANEITVNGGGSHHHHHH